SEQUENCE LISTING (83 sequences)

SEQ ID NO: 1 - protein, 218 aa, Artificial Sequence
  human and mouse sequences
DIVMTQTPLSLSVTPGQPASISCKASQSLDYEGDSDMNWYLQKPGQPPQLLIYGASNLESGVPDRFSGSGSGTDFTLKISRVEAEDVGVYYCQQSTEDPRTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 2 - protein, 449 aa, Artificial Sequence
  human and mouse sequences
QMQLVQSGPEVKKPGTSVKVSCKASGYTFTDYNVDWVRQARGQRLEWIGDINPNNGGTIYAQKFQERVTITVDKSTSTAYMELSSLRSEDTAVYYCARNYRWFGAMDHWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 15 aa, Artificial Sequence
  Mouse (Mus)
KASQSLDYEGDSDMN

SEQ ID NO: 4 - protein, 7 aa, Artificial Sequence
  Mouse (Mus)
GASNLES

SEQ ID NO: 5 - protein, 9 aa, Artificial Sequence
  Mouse (Mus)
QQSTEDPRT

SEQ ID NO: 6 - protein, 5 aa, Artificial Sequence
  Mouse (Mus)
DYNVD

SEQ ID NO: 7 - protein, 17 aa, Artificial Sequence
  Mouse (Mus)
DINPNNGGTIYAQKFQE

SEQ ID NO: 8 - protein, 10 aa, Artificial Sequence
  Mouse (Mus)
NYRWFGAMDH

SEQ ID NO: 9 - protein, 218 aa, Artificial Sequence; identical to SEQ ID NO: 1
  human and mouse sequences

SEQ ID NO: 10 - protein, 449 aa, Artificial Sequence
  human and mouse sequence
QMQLVQSGPEVKKPGTSVKVSCKASGYTFTDYNVDWVRQARGQRLEWIGDINPNSGGTIYAQKFQERVTITVDKSTSTAYMELSSLRSEDTAVYYCARNYRWFGAMDHWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 11 - protein, 17 aa, Artificial Sequence
  Mouse (Mus)
DINPNSGGTIYAQKFQE

SEQ ID NO: 12 - protein, 218 aa, Artificial Sequence; identical to SEQ ID NO: 1
  human and mouse sequence

SEQ ID NO: 13 - protein, 449 aa, Artificial Sequence
  human and mouse sequences
QMQLVQSGPEVKKPGTSVKVSCKASGYTFTDYNVDWVRQARGQRLEWIGDINPNDGGTIYAQKFQERVTITVDKSTSTAYMELSSLRSEDTAVYYCARNYRWFGAMDHWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 14 - protein, 17 aa, Artificial Sequence
  Mouse (Mus)
DINPNDGGTIYAQKFQE

SEQ ID NO: 15 - protein, 218 aa, Artificial Sequence; identical to SEQ ID NO: 1
  human and mouse sequence

SEQ ID NO: 16 - protein, 449 aa, Artificial Sequence
  human and mouse sequence
QMQLVQSGPEVKKPGTSVKVSCKASGYTFTDYNVDWVRQARGQRLEWIGDINPNQGGTIYAQKFQERVTITVDKSTSTAYMELSSLRSEDTAVYYCARNYRWFGAMDHWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 17 - protein, 17 aa, Artificial Sequence
  Mouse (Mus)
DINPNQGGTIYAQKFQE

SEQ ID NO: 18 - protein, 218 aa, Artificial Sequence; identical to SEQ ID NO: 1
  human and mouse sequences

SEQ ID NO: 19 - protein, 446 aa, Artificial Sequence
  human and mouse sequences
QMQLVQSGPEVKKPGTSVKVSCKASGYTFTDYNVDWVRQARGQRLEWIGDINPNNGGTIYAQKFQERVTITVDKSTSTAYMELSSLRSEDTAVYYCARNYRWFGAMDHWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 20 - protein, 218 aa, Artificial Sequence; identical to SEQ ID NO: 1
  human and mouse sequences

SEQ ID NO: 21 - protein, 446 aa, Artificial Sequence
  human and mouse sequences
QMQLVQSGPEVKKPGTSVKVSCKASGYTFTDYNVDWVRQARGQRLEWIGDINPNDGGTIYAQKFQERVTITVDKSTSTAYMELSSLRSEDTAVYYCARNYRWFGAMDHWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 22 - protein, 218 aa, Artificial Sequence; identical to SEQ ID NO: 1
  human and mouse sequences

SEQ ID NO: 23 - protein, 446 aa, Artificial Sequence
  human and mouse sequences
QMQLVQSGPEVKKPGTSVKVSCKASGYTFTDYNVDWVRQARGQRLEWIGDINPNSGGTIYAQKFQERVTITVDKSTSTAYMELSSLRSEDTAVYYCARNYRWFGAMDHWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 24 - protein, 218 aa, Artificial Sequence; identical to SEQ ID NO: 1
  human and mouse sequences

SEQ ID NO: 25 - protein, 446 aa, Artificial Sequence
  human and mouse sequences
QMQLVQSGPEVKKPGTSVKVSCKASGYTFTDYNVDWVRQARGQRLEWIGDINPNQGGTIYAQKFQERVTITVDKSTSTAYMELSSLRSEDTAVYYCARNYRWFGAMDHWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 26 - protein, 218 aa, Artificial Sequence; identical to SEQ ID NO: 1
  human and mouse sequences

SEQ ID NO: 27 - protein, 446 aa, Artificial Sequence
  human and mouse sequences
QMQLVQSGPEVKKPGTSVKVSCKASGYTFTDYNVDWVRQARGQRLEWIGDINPNGGGTIYAQKFQERVTITVDKSTSTAYMELSSLRSEDTAVYYCARNYRWFGAMDHWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 28 - protein, 17 aa, Artificial Sequence
  Mouse (Mus)
DINPNGGGTIYAQKFQE

SEQ ID NO: 29 - protein, 17 aa, Artificial Sequence
  Mouse (Mus)
DINPNXGGTIYXQKFXX

SEQ ID NO: 30 - protein, 5 aa, Homo sapiens
DYYWN

SEQ ID NO: 31 - protein, 16 aa, Homo sapiens
EINHNGNTNSNPSLKS

SEQ ID NO: 32 - protein, 12 aa, Homo sapiens
GYSDYEYNWFDP

SEQ ID NO: 33 - protein, 11 aa, Homo sapiens
RASQSISSYLA

SEQ ID NO: 34 - protein, 7 aa, Homo sapiens
DASNRAT

SEQ ID NO: 35 - protein, 9 aa, Homo sapiens
QQRSNWPLT

SEQ ID NO: 36 - protein, 5 aa, Homo sapiens
SYGMH

SEQ ID NO: 37 - protein, 17 aa, Homo sapiens
VIWYDGSNKYYADSVKG

SEQ ID NO: 38 - protein, 13 aa, Homo sapiens
EWAVASWDYGMDV

SEQ ID NO: 39 - protein, 12 aa, Homo sapiens
RASQSVSSSYLA

SEQ ID NO: 40 - protein, 7 aa, Homo sapiens
GASSRAT

SEQ ID NO: 41 - protein, 9 aa, Homo sapiens
QQYGSSPFT

SEQ ID NO: 42 - protein, 5 aa, Homo sapiens
DYGMS

SEQ ID NO: 43 - protein, 17 aa, Homo sapiens
GINWNGGSTYYADSVKG

SEQ ID NO: 44 - protein, 5 aa, Artificial Sequence
  Mouse (Mus)
PVGVV

SEQ ID NO: 45 - protein, 11 aa, Homo sapiens
RASQGIRSALA

SEQ ID NO: 46 - protein, 7 aa, Homo sapiens
DASSLES

SEQ ID NO: 47 - protein, 9 aa, Homo sapiens
QQFNSYPYT

SEQ ID NO: 48 - protein, 5 aa, Homo sapiens
GYYWS

SEQ ID NO: 49 - protein, 16 aa, Homo sapiens
EINHRGNTNCNPSLKS

SEQ ID NO: 50 - protein, 12 aa, Homo sapiens
GYDILTGYYEDS

SEQ ID NO: 51 - protein, 11 aa, Homo sapiens
RASQSVSSYLA

SEQ ID NO: 52 - protein, 7 aa, Homo sapiens
NASNRAT

SEQ ID NO: 53 - protein, 9 aa, Homo sapiens
QQRSNWPLT

SEQ ID NO: 54 - protein, 5 aa, Homo sapiens
EVSMH

SEQ ID NO: 55 - protein, 17 aa, Homo sapiens
GFDPEDGETIYAQKFQG

SEQ ID NO: 56 - protein, 11 aa, Homo sapiens
AFVVVVAASDY

SEQ ID NO: 57 - protein, 11 aa, Homo sapiens
RASQSVSSYLA

SEQ ID NO: 58 - protein, 7 aa, Artificial Sequence
  Mouse (Mus)
DASNRAT

SEQ ID NO: 59 - protein, 9 aa, Homo sapiens
QQRSNWPWT

SEQ ID NO: 60 - protein, 5 aa, Homo sapiens
SYGMH

SEQ ID NO: 61 - protein, 17 aa, Homo sapiens
VIWYDGSNKYYADSVKG

SEQ ID NO: 62 - protein, 14 aa, Homo sapiens
DPHCSSTNCYLFDY

SEQ ID NO: 63 - protein, 11 aa, Homo sapiens
RASQSVSSYLA

SEQ ID NO: 64 - protein, 7 aa, Homo sapiens
DASNRAT

SEQ ID NO: 65 - protein, 9 aa, Homo sapiens
QQRSNWPIT

SEQ ID NO: 66 - protein, 8 aa, Artificial Sequence
  Mouse (Mus)
GFSLTAYG

SEQ ID NO: 67 - protein, 7 aa, Artificial Sequence
  Mouse (Mus)
IWDDGST

SEQ ID NO: 68 - protein, 10 aa, Artificial Sequence
  Mouse (Mus)
AREGDVAFDY

SEQ ID NO: 69 - protein, 12 aa, Artificial Sequence
  Mouse (Mus)
QSLLNGSNQKNY

SEQ ID NO: 70 - protein, 3 aa, Artificial Sequence
  Mouse (Mus)
FAS

SEQ ID NO: 71 - protein, 9 aa, Artificial Sequence
  Mouse (Mus)
LQHFGTPPT

SEQ ID NO: 72 - protein, 5 aa, Artificial Sequence
  Mouse (Mus)
AYGVN

SEQ ID NO: 73 - protein, 16 aa, Artificial Sequence
  Mouse (Mus)
MIWDDGSTDYDSALKS

SEQ ID NO: 74 - protein, 8 aa, Artificial Sequence
  Mouse (Mus)
EGDVAFDY

SEQ ID NO: 75 - protein, 17 aa, Artificial Sequence
  Mouse (Mus)
KSSQSLLNPSNQKNYLA

SEQ ID NO: 76 - protein, 7 aa, Artificial Sequence
  Mouse (Mus)
FASTRDS

SEQ ID NO: 77 - protein, 9 aa, Artificial Sequence
  Mouse (Mus)
LQHFGTPPT

SEQ ID NO: 78 - protein, 525 aa, Homo sapiens
MWEAQFLGLLFLQPLWVAPVKPLQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

SEQ ID NO: 79 - protein, 533 aa, Macaca fascicularis
  misc_feature (74)..(74): Xaa can be any naturally occurring amino acid
MWEAQFLGLLFLQPLWVAPVKPPQPGAEISVVWAQEGAPAQLPCSPTIPLQDLSLLRRAGVTWQHQPDSGPPAXAPGHPPVPGHRPAAPYSWGPRPRRYTVLSVGPGGLRSGRLPLQPRVQLDERGRQRGDFSLWLRPARRADAGEYRATVHLRDRALSCRLRLRVGQASMTASPPGSLRTSDWVILNCSFSRPDRPASVHWFRSRGQGRVPVQGSPHHHLAESFLFLPHVGPMDSGLWGCILTYRDGFNVSIMYNLTVLGLEPATPLTVYAGAGSRVELPCRLPPAVGTQSFLTAKWAPPGGGPDLLVAGDNGDFTLRLEDVSQAQAGTYICHIRLQGQQLNATVTLAIITVTPKSFGSPGSLGKLLCEVTPASGQEHFVWSPLNTPSQRSFSGPWLEAQEAQLLSQPWQCQLHQGERLLGAAVYFTELSSPGAQRSGRAPGALRAGHLPLFLILGVLFLLLLVTGAFGFHLWRRQWRPRRFSALEQGIHPPQAQSKIEELEQEPELEPEPELERELGPEPEPGPEPEPEQL

SEQ ID NO: 80 - DNA, 30 bp, Artificial Sequence
  NFAT transcriptional response element sequence
ggaggaaaaactgtttcatacagaaggcgt

SEQ ID NO: 81 - protein, 529 aa, Artificial Sequence
  VEGF leader sequence-human LAG3 fusion
MNFLLSWVHWSLALLLYLHHAKWSQALQPGAEVPVVWAQEGAPAQLPCSPTIPLQDLSLLRRAGVTWQHQPDSGPPAAAPGHPLAPGPHPAAPSSWGPRPRRYTVLSVGPGGLRSGRLPLQPRVQLDERGRQRGDFSLWLRPARRADAGEYRAAVHLRDRALSCRLRLRLGQASMTASPPGSLRASDWVILNCSFSRPDRPASVHWFRNRGQGRVPVRESPHHHLAESFLFLPQVSPMDSGPWGCILTYRDGFNVSIMYNLTVLGLEPPTPLTVYAGAGSRVGLPCRLPAGVGTRSFLTAKWTPPGGGPDLLVTGDNGDFTLRLEDVSQAQAGTYTCHIHLQEQQLNATVTLAIITVTPKSFGSPGSLGKLLCEVTPVSGQERFVWSSLDTPSQRSFSGPWLEAQEAQLLSQPWQCQLYQGERLLGAAVYFTELSSPGAQRSGRAPGALPAGHLLLFLILGVLSLLLLVTGAFGFHLWRRQWRPRRFSALEQGIHPPQAQSKIEELEQEPEPEPEPEPEPEPEPEPEQL

SEQ ID NO: 82 - DNA, 1590 bp, Artificial Sequence
  VEGF leader sequence-human LAG3 fusion
atgaactttctgctgagctgggtgcactggtccctggccctgctgctgtacctgcaccacgctaagtggtcccaggctctgcagcctggcgctgaagtgcctgtcgtgtgggctcaggaaggcgctcctgcccagctgccttgcagccctacaatcccactgcaggacctgagcctgctgagaagggctggcgtgacatggcagcaccagcctgattctggacctcctgctgctgctcctggacacccactggctccaggacctcacccagctgctccaagctcttggggccccagacctagaagatacaccgtgctgtctgtgggccctggcggcctgagatctggtagactgcctctgcagccaagagtgcagctggacgagagaggcagacagaggggcgacttcagcctgtggctgaggccagctagaagggccgatgccggcgagtacagagccgccgtgcatctgagagacagagccctgagctgcagactgagactgaggctgggccaggccagcatgacagctagccctccaggcagcctgagagccagcgactgggtcatcctgaactgcagcttcagcagacccgacagacccgccagcgtgcactggttcagaaacagaggccagggcagagtgcccgtgcgcgagtctcctcaccaccacctggccgagagctttctgttcctgccacaggtgtcccccatggactctggcccttggggctgcatcctgacctacagggacggcttcaacgtgtccatcatgtacaacctgaccgtgctgggcctggaaccccctacccctctgacagtgtacgctggcgctggctctagagtgggcctgccttgtagactgccagccggcgtgggcaccagaagctttctgaccgccaagtggacacctcccggcggaggacctgatctgctggtcaccggcgacaacggcgacttcacactgagactggaagatgtgtcccaggcccaggccggcacctacacctgtcacatccatctgcaggaacagcagctgaacgccaccgtgaccctggccatcatcaccgtgacacccaagagcttcggcagccctggctctctgggcaagctgctgtgtgaagtgacccccgtgtccggccaggaaagattcgtctggtccagcctggacacacccagccagagaagcttcagcggcccttggctggaagcccaggaagcacagctgctgagccagccttggcagtgccagctgtaccagggcgagagactgctgggcgctgccgtgtacttcaccgagctgagtagccctggcgcccagagaagtggtagagcacctggtgccctgcctgccggccatctgctgctgtttctgatcctgggcgtgctgtccctgctcctgctcgtgacaggcgctttcggcttccacctgtggcggagacagtggcggcctagaagattcagcgccctggaacagggcatccaccctccacaggcccagagcaagatcgaggaactggaacaggaacccgagcccgagcctgagcccgaaccagaacctgagcctgaacctgagccagagcagctgtga

SEQ ID NO: 83 - DNA, 9 bp, Artificial Sequence
  Kozak sequence
gccgccacc

(19) United States
(12) Patent Application Publication
(10) Pub. No.: US 2017/0097333 A1
(43) Pub. Date: Apr. 6, 2017

(54) CELL BASED ASSAY TO MEASURE THE T-CELL STIMULATING CAPACITY OF ANTI-LAG3 ANTIBODIES AND OTHER AGENTS

(71) Applicant: Merck Sharp & Dohme Corp., Rahway, NJ (US)

(72) Inventors: Bhagyashree Bhagwat, Cupertino, CA (US); Aarron Willingham, Mountain View, CA (US)

(73) Assignee: Merck Sharp & Dohme Corp., Rahway, NJ (US)

(21) Appl. No.: 15/278,358

(22) Filed: Sep. 28, 2016

Related U.S. Application Data
(60) Provisional application No. 62/233,652, filed on Sep. 28, 2015.

(51) Int. Cl. G01N 33/50; C07K 14/705; C07K 14/725; C12N 15/86

(52) U.S. Cl. CPC G01N 33/505; C12N 15/86; C07K 14/70503; C07K 14/7051; C07K 14/70532; G01N 2333/7051; G01N 2500/10

(57) ABSTRACT

The present invention includes a human LAG3 functional assay using a Jurkat T-cell lymphoma cell line engineered to overexpress LAG3 at an optimal level relative to CD3. The assay is useful, for example, for determining the immunostimulatory properties of LAG3 modulators (e.g., inhibitors or activators). The optimized LAG3/CD3 ratio ensures expression of optimal receptor components on the T-cell and, thus, superior assay sensitivity. Immunostimulation of the T-cells can be measured, for example, by following cytokine (e.g., IL-2) production. The optimized T-cell line forms part of the present invention along with compositions generated with use of the assay.

This Application claims the benefit of U.S. Provisional Patent Application No. 62/233,652, filed Sep. 28, 2015; which is herein incorporated by reference in its entirety.

### FIELD OF THE INVENTION

The present invention relates to methods and compositions for evaluating the immunostimulatory properties of a test substance.

### BACKGROUND OF THE INVENTION

LAG3 (Lymphocyte Activation Gene-3) is an inhibitory receptor that is structurally similar to CD4, contains four Ig-like domains and binds to MHC class II molecules. LAG3 has been shown to negatively regulate T-cell activation and proliferation as well as to be co-expressed on tumor-infiltrating lymphocytes with other inhibitory receptors and is indicative of a highly exhausted T-cell phenotype.

With the recent generation of various LAG3 therapeutic agents, there is a great interest in developing high sensitivity assays for accurately determining the ability of such agents to stimulate T-cells.

### SUMMARY OF THE INVENTION

The present invention provides an isolated human or cynomolgous monkey T-cell which express LAG3 (e.g., human LAG3) and CD3 (e.g., human CD3) at a ratio of about 2/1 (LAG3/CD3) or lower and, optionally, PD-1 (e.g., human PD-1). In an embodiment of the invention, wherein the cell, when stained with Ab6 (e.g., IgG4/x) labeled with a fluorescent label having an excitation maximum of about 652 nm and an emission maximum of about 672 nm (e.g., wherein the label has a molar extinction coefficient (M⁻¹ cm⁻¹) and/or a molecular weight of about 1066 g/mole; for example, wherein the label is DyLight650), or with an anti-LAG3 antibody comprising a light chain comprising the amino acid sequence set forth in SEQ ID NO: 84 and a heavy chain comprising the amino acid sequence set forth in SEQ ID NO: 85 (e.g., IgG1/x) labeled with a fluorescent label having an excitation maximum of about 652 nm and an emission maximum of about 672 nm (e.g., wherein the label has a molar extinction coefficient (M⁻¹ cm⁻¹) and/or a molecular weight of about 1066 g/mole; for example, wherein the label is DyLight650), along with maHuCD3 pacblue; exhibits fluorescent intensities indicative of an expression level of LAG3 and CD3 which is at a ratio of about 2/1 or lower. In an embodiment of the invention, the T-cell is a Jurkat cell. In an embodiment of the invention, the fluorescent intensity of the anti-LAG3 is relative to that of a control antibody with the same label (e.g., labeled trastuzumab) and the intensity of the anti-CD3 is relative to that of a control antibody with the same label (e.g., mIgG1-pacblue). In an embodiment of the invention, the fluorescent intensity is measured by FACS. The present invention also provides a composition comprising such a human or cynomolgous monkey T-cell and a human or cynomolgous monkey antigen-presenting cell (APC), optionally, wherein the APC expresses PD-L1. In an embodiment of the invention, the APC is a Raji, CH12.1 or DAP-DR1 cell. The composition may also include other items such as a T-cell activating agent (e.g., Staphylococcal enterotoxin, Staphylococcal enterotoxin B (e.g., which is not Staphylococcal enterotoxin B (SEB) if the T-cell is a Jurkat cell), Staphylococcal enterotoxin A (SEA), Staphylococcal enterotoxin D (SED) or Staphylococcal enterotoxin E (SEE)). In an embodiment of the invention, the composition also comprises an agent that inhibits LAG3, such as an anti-LAG3 antibody or antigen-binding fragment or a LAG3 peptide (e.g., any of those set forth below under “Test Substances”). The present invention also provides a composition including human or cynomolgous monkey T-cells and human or cynomolgous monkey antigen-presenting cells that are at a ratio of about 1/1, 2/1, 4/1, 8/1, 16/1 or 32/1 (T-cells/APCs).

The present invention also provides a method for determining if or to what extent a test substance stimulates T-cells comprising contacting a composition that comprises human or cynomolgous monkey T-cells which express LAG3 and CD3 at a ratio of about 2/1 (LAG3/CD3) or lower and human or cynomolgous monkey antigen-presenting cells, in the presence of a T-cell activating agent, with the test substance; and determining whether the T-cells are activated; with the proviso that the T-cell activating agent is not Staphylococcal enterotoxin B (SEB) if the T-cell is a Jurkat cell. In an embodiment of the invention, IL-2, interferon gamma (IFNγ), IL-8 and/or tumor necrosis factor alpha (TNFα) produced from the T-cells is measured to determine whether the T-cells are activated; wherein the T-cells are determined to be activated if production of IL-2, IL-8, IFNγ and/or TNFα by the T-cells increases in the presence of the test substance relative to in the absence of the test substance. The level of cytokine production by the T-cells indicates the extent to which the cells are activated. In an embodiment of the invention, expression of a reporter gene that is operably associated with an nuclear factor of activated T-cells response element (NFAT-RE) is measured to determine whether the T-cells are activated; wherein the T-cells are determined to be activated if expression of the reporter gene increases in the presence of the test substance relative to in the absence of the test substance. The level of reporter expression indicates the extent to which the T-cells are activated. In an embodiment of the invention, the human or cynomolgous monkey T-cells and human or cynomolgous monkey antigen-presenting cells are at a ratio of about 1/1, 2/1, 4/1, 8/1, 16/1 or 32/1.

In addition, the present invention provides a method for making the composition of the present invention that comprises T-cells (e.g., Jurkat cells) and antigen-presenting cells (e.g., Raji cells) comprising combining the T-cells and the antigen-presenting cells. Any composition that is the product of such a method is part of the present invention. The scope of the present invention also includes a method for making a T-cell of the present invention comprising introducing a polynucleotide encoding LAG3 into one or more T-cells (e.g., Jurkat cells), determining which T-cells express LAG3 and CD3 at a ratio of about 2/1 (LAG3/CD3) or lower and selecting one or more T-cells exhibiting said ratio. A T-cell that is the product of such a method is part of the present invention.

### BRIEF DESCRIPTION OF THE FIGURES

FIG. 1. SED- and SEE-mediated stimulation of Jurkat cells co-cultured with Raji cells evaluated using an NFAT transcriptional element linked to a luciferase reporter gene.

FIG. 2 (A)-(D). (A) FACS analysis of LAG3 transduced Jurkat cells stained with anti-LAG3 or control antibody; (B) FACS analysis of LAG3 transduced Jurkat cells sorted for higher LAG3 expression and then stained with anti-LAG3 or control antibody; (C-D) FACS analysis of Jurkat parental cells, and LAG3 or vector transduced Jurkat cells stained with anti-LAG3 or anti-CD3.

FIG. 3. Analysis of quantity of LAG3 and CD3 expression by 67 different clones of Jurkat cells transduced with human LAG3 and stained with anti-LAG3 or anti-CD3. Clones included G3, F5, E6, A1, D7, H3, G10 and F11.

FIG. 4 (A)-(D). Expression of IL-2 by Jurkat clone G10 or parental non-transfected Jurkat cells co-cultured with Raji cells and stimulated with (A) SEA, (B) SED or (C) SEE. (D) Expression of IL-2 by Jurkat clone G10 stimulated with increasing concentrations of SEA in the presence of anti-MHC Class II antibody (10 μg/ml) or of no antibody.

FIG. 5. Analysis of Jurkat clones G10 and F11 stained with anti-LAG3 (boxes on left). IL-2 production by Jurkat clones G10 and F11 co-cultured with Raji cells in the presence of 100 ng/ml of SED.

FIG. 6. IL2 production (electrochemilluminescent signal) by G10 Jurkat clones in the presence of Raji cells (at ratios of 1, 2, 4, 8, 16 or 32 (G10/Raji)), 500 ng/ml SEA, and in the presence or absence of anti-LAG3 antibody (10 μg/ml of humanized anti-LAG3 antibody, (VH4/VL1) IgG4 S228P/Kappa (PX)).

FIG. 7 (A)-(B). Comparison of SEA and SED antigen stimulation in Jurkat assay. Jurkat cells stimulated with (A) 500 ng/ml SEA or (B) 50 ng/ml SED in the presence of Raji cells and increasing concentrations of anti-LAG3 (35AHK: humanized anti-human LAG3 VH6 N55Q/VL3, IgG4 S228P/Kappa, 00AGJ: humanized anti-human LAG3 VH4/VL1, IgG4 S228P/Kappa or 83AFM: anti-human LAG3 human IgG1/mouse constant kappa) or anti-CTLA4 (08AFG: human anti-human CTLA4 (IgG1/Kappa)). Jurkat T-cell secretion of IL-2 monitored.

FIG. 8. Comparison of clone G10 and F11 performance in Jurkat assay. Jurkat cells (clone G10 or clone F11) stimulated with 100 ng/ml of SED in the presence of Raji cells and increasing concentrations of anti-LAG3 (Ab6; humanized light chain (VL3)/Kappa (PX) and humanized heavy chain VH6 N55D/VL3) IgG4 S228P/Kappa (PX)). T-cell secretion of IL-2 monitored.

FIG. 9. Comparison of humanized anti-human LAG3 (51AHH, Ab6; VH6 N55D/VL3) IgG4 S228P/Kappa), mouse anti-human LAG3 (87AHE; IgG2a/Kappa) and human anti-human CTLA4 (IgG1/Kappa) in Jurkat assay. Jurkat cells (clone G10) stimulated (50 ng/ml SED) in the presence of Raji cells and increasing concentrations of anti-LAG3 or anti-CTLA4. T-cell secretion of IL-2 monitored.

FIG. 10 (A)-(B). (A) FACS analysis of LAG3 and CD3 expression in Jurkat cells containing a cynomolgus monkey LAG3 transgene. (B) Comparison of two samples of Jurkat cells expressing different levels of cynomolgous LAG3 and human CD3 in Jurkat assay. Secretion of IL-2 was monitored from two samples of Jurkat cells stimulated with SED antigen (boxes: approximately equivalent LAG3 and CD3 expression levels; circles: pool of cynomolgus LAG-3 Jurkat cells with a broad expression range of LAG3:CD3) in the presence of Raji cells of increasing concentrations of humanized anti-human LAG3 VH6 N55D/VL3, IgG4 S228P/Kappa (51AHH, Ab6).

FIG. 11 (A)-(B). Comparison of three different anti-LAG3 antibodies (51AHH, Ab6: humanized anti-human LAG3 VH6 N55D/VL3, IgG4 S228P/Kappa, 45AHR: human anti-human LAG3, IgG4 S228P/Kappa, 51AHR: human anti-human LAG3, IgG4 S228P/kappa) and two other anti-LAG3 antibodies) in human and cynomolgous T-cell assay. (A) Jurkat cells expressing human LAG3 or (B) Jurkat cells expressing cynomolgous LAG3 were stimulated by antigen (SED) in the presence of Raji cells and increasing concentrations of anti-LAG3 antibody and secretion of IL-2 was monitored. 85AER (humanized anti-RSV, hinge mutation S228P IgG4) is a control and has no activity in these assays.

FIG. 12 (A)-(B). (A) Analysis of LAG3 and CD3 expression in Jurkat cells transduced with lentivirus constructs containing human LAG3 and NFAT-luciferase. LAG3 and CD3 expression was evaluated and a sub-population of cells was sorted and selected based on an approximate 1:1 ratio of expression. (B) Comparison of anti-LAG3 and anti-RSV antibody performance in Jurkat cells assay. The sorted pool of Jurkat LAG3 NFAT-luciferase cells were co-cultured with Raji cells and stimulated with SED or SEE and cell luminescence from luciferase was monitored in the presence of increasing concentrations of anti-LAG3 (Ab6; humanized light chain (VL3)/Kappa (PX) and humanized heavy chain VH6 N55D/VL3) IgG4 S228P/Kappa (PX)) or anti-RSV (respiratory syncytial virus).

FIG. 13 (A)-(D). Evaluation of secretion of various cytokines in Jurkat cell assay. Jurkat cells were stimulated by antigen (SED) in the presence of Raji cells and anti-LAG3 or anti-CTLA4 (51AHH, Ab6: humanized anti-human LAG3 VH6 N55D/VL3, IgG4 S228P/Kappa, 26AHK: humanized anti-human LAG3 VH6 N55S/VL3, IgG4 S228P/Kappa, 35AHK: humanized anti-human LAG3 VH6 N55Q/VL3, IgG4 S228P/Kappa, 87AHE: Mouse anti-human LAG3 IgG2a/Kappa and 08AFG: human anti-human CTLA4 IgG1/Kappa). Secretion of (A) IL-2, (B) interferon-gamma, (C) IL-8 or (D) TNF-alpha from the Jurkat cells was monitored.

FIG. 14. Impact of combining anti-human LAG3 and anti-human PD-1. Effect of anti-human LAG3, anti-human PD-1 or both on IL-2 expression from T-cells expressing human LAG3, human CD3 and human PD-1 in the presence of APCs expressing endogenous MHC class II and human PD-L1.

### DETAILED DESCRIPTION OF THE INVENTION

A human LAG3 functional assay was developed using a Jurkat T-cell lymphoma line engineered to overexpress LAG3 and co-cultured with a Raji B-cell lymphoma line in the presence of sub-optimal levels of staphylococcal enterotoxins (e.g., SEA, SED or SEE). Treatment with anti-LAG3 mAbs leads to a dose-dependent release of LAG3 mediated repression, an approximate 10-fold increase in IL-2 levels, and sufficient assay reproducibility to calculate EC50 values. In addition to IL-2, IFNgamma, IL-8 and TNFalpha have also been shown to increase in a dose-responsive fashion upon treatment with anti-LAG3. Furthermore, LAG3 signaling through NFAT and modulation by anti-LAG3 has been demonstrated using a luciferase reporter construct introduced into the Jurkat bioassay system—providing the first report that LAG3 signals through NFAT. This Jurkat LAG3 bioassay is a fully human assay system that represents a novel combination of stimulation factors and cell engineering to ensure optimal receptor components to enable LAG3 repression of super-antigen mediated stimulation of the MHC class II and T cell receptor complex and to measure relief of LAG3 repression by treatment with anti-LAG3. This Jurkat LAG3 bioassay will have utility for CMC potency release, antibody screening, quality control and bioactivity testing. Assessment of LAG3 mechanism of action and signal transduction, and cross-species potency comparisons were conducted by substituting a cynomolgus monkey LAG3 trans gene. The assay of the present invention may also be used to determine the T-cell activation caused by a combination of LAG3 modulator and a modulator of any other protein expressed by the T-cells (e.g., endogenously or due to introduction of a gene expressing the other protein), such as an immunomodulatory receptor such as PD1 (e.g., human PD1).

“Anti-LAG3” refers to an antibody or antigen-binding fragment thereof that binds specifically to LAG3.

### Test Substances

Any test substance can be analyzed using the methods of the present invention so as to determine whether the substance stimulates T-cells. In an embodiment of the invention, the test substance can be a LAG3 inhibitor or a LAG3 agonist such as an anti-LAG3 antibody or antigen-binding fragment thereof. A LAG3 inhibitor is a substance that inhibits one or more activities of LAG3 such as, for example, if the substance inhibits LAG3 binding to MHC class inhibits the inhibition of T-cell activation, e.g., leading to LAG3-dependent stimulation of T-cells (e.g., stimulates cytokine production from T-cells), for example, in the presence of a T-cell stimulatory substance; or inhibits LAG3 homodimerization.

A LAG3 agonist may agonize a LAG3 activity, for example, by stimulating LAG3 binding to MHC class II, stimulating LAG3 homodimerization or stimulating LAG3-dependent inhibition of T-cells. In an embodiment of the invention, a cytotoxic anti-LAG3 antibody or antigen-binding fragment thereof that depletes T-cells may be considered a LAG3 agonist since it leads to reduced T-cell activity. An example of such an antibody is IMP731.

Antagonist anti-LAG3 antibodies and fragments thereof include those set for the below: Ab1, Ab2, Ab3, Ab4, Ab5, Ab6, Ab7, Ab8 or Ab9. The soluble extracellular domain of LAG3 fused to an immunoglobulin polypeptide, IMP321, is an example of another LAG3 antagonist.
Ab1: humanized light chain 45AGX Humanized×[LAG3_H] mAb (LB145.22D2.E1.D1 (VL3)) Kappa (PX) (or the variable domain thereof) and humanized heavy chain 53AHH Humanized×[LAG3_H] mAb (LB145.22D2.E1.D1 VH6) IgG1/Kappa (PX) (or the variable domain thereof); for example comprising:

a light chain immunoglobulin comprising the amino
acid sequence:
(SEQ ID NO: 1)
DIVMTQTPLSLSVTPGQPASISCKASQSLDYEGDSDMNWYLQKPGQPPQL
LIYGASNLESGVPDRFSGSGSGTDFTLKISRVEAEDVGVYYCQQSTEDPR
TEGGGTKVEIKRTVAAPSVFIFPPSDEQLKSGTASVVCLLNNEYPREAKV
QWKVDNALQSGNSQESVTEQDSKDSTYSLSSTLTLSKADYEKHKVYACEV
THQGLSSPVTKSFNRGEC;
and
a heavy chain immunoglobulin comprising the amino
acid sequence:
(SEQ ID NO: 2)
QMQLVQSGPEVKKPGTSVKVSCKASGYTFTDYNVDWVRQARGQRLEWIGD
INPNNGGTIYAQKFQERVTITVDKSTSTAYMELSSLRSEDTAVYYCARNY
RWFGAMDHWGQGTTVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLEPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPGK;
or
a light chain immunoglobulin variable domain
comprising the amino acid sequence:
(amino acids 21-131 or SEQ ID NO: 1 (CDRs
underscored))
DIVMTQTPLSLSVTPGQPASISCKASQSLDYEGDSDMNWYLQKPGQPPQL
LIYGASNLESGVPDRFSGSGSGTDFTLKISRVEAEDVGVYYCQQSTEDPR
TFGGGTKVEIK;
and
a heavy chain immunoglobulin variable domain
comprising the amino acid sequence:
(amino acids 1-119 of SEQ ID NO: 2 (CDRs
underscored))
QMQLVQSGPEVKKPGTSVKVSCKASGYTFTDYNVDWVRQARGQRLEWIGD
INPNNGGTIYAQKFQERVTITVDKSTSTAYMELSSLRSEDTAVYYCARNY
RWFGAMDHWGQGTTVTVSS,
or
comprising the CDRs:
CDR-L1:
(SEQ ID NO: 3)
KASQSLDYEGDSDMN;
CDR-L2:
(SEQ ID NO: 4)
GASNLES;
CDR-L3:
(SEQ ID NO: 5)
QQSTEDPRT;
CDR-H1:
(SEQ ID NO: 6)
DYNVD;
CDR-H2:
(SEQ ID NO: 7)
DINPNNGGTIYAQKFQE;
and
CDR-H3:
(SEQ ID NO: 8)
NYRWFGAMDH

Ab2: humanized light chain 45AGX Humanized×[LAG3_H] mAb (LB145.22D2.E1.D1 (VL3)) Kappa (PX) (or the variable domain thereof) and humanized heavy chain 56AHH Humanized×[LAG3_H] mAb (LB145.22D2.E1.D1 VH6 N55S) IgG1/Kappa (PX) (or the variable domain thereof); for example: comprising:

a light chain immunoglobulin comprising the amino
acid sequence:
(SEQ ID NO: 9)
DIVMTQTPLSLSVTPGQPASISCKASQSLDYEGDSDMNWYLQKPGQPPQL
LIYGASNLESGVPDRFSGSGSGTDFTLKISRVEAEDVGVYYCQQSTEDPR
TEGGGTKVEIKRTVAAPSVFIFPPSDEQLKSGTASVVCLLNNEYPREAKV
QWKVDNALQSGNSQESVTEQDSKDSTYSLSSTLTLSKADYEKHKVYACEV
THQGLSSPVTKSFNRGEC;
and
a heavy chain immunoglobulin comprising the amino
acid sequence:
(SEQ ID NO: 10)
QMQLVQSGPEVKKPGTSVKVSCKASGYTFTDYNVDWVRQARGQRLEWIGD
INPNSGGTIYAQKFQERVTITVDKSTSTAYMELSSLRSEDTAVYYCARNY
RWFGAMDHWGQGTTVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLEPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPGK;
or
a light chain immunoglobulin variable domain
comprising the amino acid sequence:
(amino acids 21-131 or SEQ ID NO: 9 (CDRs
underscored))
DIVMTQTPLSLSVTPGQPASISCKASQSLDYEGDSDMNWYLQKPGQPPQL
LIYGASNLESGVPDRFSGSGSGTDFTLKISRVEAEDVGVYYCQQSTEDPR
TFGGGTKVEIK;
and
a heavy chain immunoglobulin variable domain
comprising the amino acid sequence:
(amino acids 1-119 of SEQ ID NO: 10 (CDRs
underscored))
QMQLVQSGPEVKKPGTSVKVSCKASGYTFTDYNVDWVRQARGQRLEWIGD
INPNSGGTIYAQKFQERVTITVDKSTSTAYMELSSLRSEDTAVYYCARNY
RWFGAMDHWGQGTTVTVSS,
or
comprising the CDRs:
CDR-L1:
(SEQ ID NO: 3)
KASQSLDYEGDSDMN;
CDR-L2:
(SEQ ID NO: 4)
GASNLES;
CDR-L3:
(SEQ ID NO: 5)
QQSTEDPRT;
CDR-H1:
(SEQ ID NO: 6)
DYNVD;
CDR-H2:
(SEQ ID NO: 11)
DINPNSGGTIYAQKFQE;
and
CDR-H3:
(SEQ ID NO: 8)
NYRWFGAMDH

Ab3: humanized light chain 45AGX Humanized×[LAG3_H] mAb (LB145.22D2.E1.D1 (VL3)) Kappa (PX) (or the variable domain thereof) and humanized heavy chain 54AHH Humanized×[LAG3_H] mAb (LB145.22D2.E1.D1 VH6 N55D) IgG1/Kappa (PX) (or the variable domain thereof); for example comprising:

a light chain immunoglobulin comprising the amino
acid sequence:
(SEQ ID NO: 12)
DIVMTQTPLSLSVTPGQPASISCKASQSLDYEGDSDMNWYLQKPGQPPQL
LIYGASNLESGVPDRFSGSGSGTDFTLKISRVEAEDVGVYYCQQSTEDPR
TEGGGTKVEIKRTVAAPSVFIFPPSDEQLKSGTASVVCLLNNEYPREAKV
QWKVDNALQSGNSQESVTEQDSKDSTYSLSSTLTLSKADYEKHKVYACEV
THQGLSSPVTKSFNRGEC
a heavy chain immunoglobulin comprising the amino
acid sequence:
(SEQ ID NO: 13)
QMQLVQSGPEVKKPGTSVKVSCKASGYTFTDYNVDWVRQARGQRLEWIGD
INPNDGGTIYAQKFQERVTITVDKSTSTAYMELSSLRSEDTAVYYCARNY
RWFGAMDHWGQGTTVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLEPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPGK;
or
a light chain immunoglobulin variable domain
comprising the amino acid sequence:
(amino acids 21-131 or SEQ ID NO: 12 (CDRs
underscored))
DIVMTQTPLSLSVTPGQPASISCKASQSLDYEGDSDMNWYLQKPGQPPQL
LIYGASNLESGVPDRFSGSGSGTDFTLKISRVEAEDVGVYYCQQSTEDPR
TFGGGTKVEIK;
and
a heavy chain immunoglobulin variable domain
comprising the amino acid sequence:
(amino acids 1-119 of SEQ ID NO: 13 (CDRs
underscored))
QMQLVQSGPEVKKPGTSVKVSCKASGYTFTDYNVDWVRQARGQRLEWIGD
INPNDGGTIYAQKFQERVTITVDKSTSTAYMELSSLRSEDTAVYYCARNY
RWFGAMDHWGQGTTVTVSS,
or
comprising the CDRs:
CDR-L1:
(SEQ ID NO: 3)
KASQSLDYEGDSDMN;
CDR-L2:
(SEQ ID NO: 4)
GASNLES;
CDR-L3:
(SEQ ID NO: 5)
QQSTEDPRT;
CDR-H1:
(SEQ ID NO: 6)
DYNVD;
CDR-H2:
(SEQ ID NO: 14)
DINPNDGGTIYAQKFQE;
and
CDR-H3:
(SEQ ID NO: 8)
NYRWFGAMDH

Ab4: humanized light chain 45AGX Humanized×[LAG3_H] mAb (LB145.22D2.E1.D1 (VL3)) Kappa (PX) (or the variable domain thereof) and humanized heavy chain 52AHH Humanized×[LAG3_H] mAb (LB145.22D2.E1.D1 VH6 N55Q) IgG1/Kappa (PX) (or the variable domain thereof); for example comprising:

a light chain immunoglobulin comprising the amino
acid sequence:
(SEQ ID NO: 15)
DIVMTQTPLSLSVTPGQPASISCKASQSLDYEGDSDMNWYLQKPGQPPQL
LIYGASNLESGVPDRFSGSGSGTDFTLKISRVEAEDVGVYYCQQSTEDPR
TEGGGTKVEIKRTVAAPSVFIFPPSDEQLKSGTASVVCLLNNEYPREAKV
QWKVDNALQSGNSQESVTEQDSKDSTYSLSSTLTLSKADYEKHKVYACEV
THQGLSSPVTKSFNRGEC;
and
a heavy chain immunoglobulin comprising the amino
acid sequence:
(SEQ ID NO: 16)
QMQLVQSGPEVKKPGTSVKVSCKASGYTFTDYNVDWVRQARGQRLEWIGD
INPNQGGTIYAQKFQERVTITVDKSTSTAYMELSSLRSEDTAVYYCARNY
RWFGAMDHWGQGTTVTVSSASTKGPSVFPLAPSSKSTSGGTAALGCLVKD
YFPEPVTVSWNSGALTSGVHTFPAVLQSSGLYSLSSVVTVPSSSLGTQTY
ICNVNHKPSNTKVDKKVEPKSCDKTHTCPPCPAPELLGGPSVFLEPPKPK
DTLMISRTPEVTCVVVDVSHEDPEVKFNWYVDGVEVHNAKTKPREEQYNS
TYRVVSVLTVLHQDWLNGKEYKCKVSNKALPAPIEKTISKAKGQPREPQV
YTLPPSRDELTKNQVSLTCLVKGFYPSDIAVEWESNGQPENNYKTTPPVL
DSDGSFFLYSKLTVDKSRWQQGNVFSCSVMHEALHNHYTQKSLSLSPGK;
or
a light chain immunoglobulin variable domain
comprising the amino acid sequence:
(amino acids 21-131 of SEQ ID NO: 15 (CDRs
underscored))
DIVMTQTPLSLSVTPGQPASISCKASQSLDYEGDSDMNWYLQKPGQPPQL
LIYGASNLESGVPDRFSGSGSGTDFTLKISRVEAEDVGVYYCQQSTEDPR
TFGGGTKVEIK;
and
a heavy chain immunoglobulin variable domain
comprising the amino acid sequence:
(amino acids 1-119 or SEQ ID NO: 16 (CDRs
underscored))
QMQLVQSGPEVKKPGTSVKVSCKASGYTFTDYNVDWVRQARGQRLEWIGD
INPNQGGTIYAQKFQERVTITVDKSTSTAYMELSSLRSEDTAVYYCARNY
RWFGAMDHWGQGTTVTVSS,
or
comprising the CDRs:
CDR-L1:
(SEQ ID NO: 3)
KASQSLDYEGDSDMN;
CDR-L2:
(SEQ ID NO: 4)
GASNLES;
CDR-L3:
(SEQ ID NO: 5)
QQSTEDPRT;
CDR-H1:
(SEQ ID NO: 6)
DYNVD;
CDR-H2:
(SEQ ID NO: 17)
DINPNQGGTIYAQKFQE;
and
CDR-H3:
(SEQ ID NO: 8)
NYRWFGAMDH

Ab5: humanized light chain 45AGX Humanized×[LAG3_H] mAb (LB145.22D2.E1.D1 (VL3)) Kappa (PX) (or the variable domain thereof) and humanized heavy chain 57AHH Humanized×[LAG3_H] mAb (LB145.22D2.E1.D1 VH6) IgG4 S228P (PX) (or the variable domain thereof); for example comprising:

a light chain immunoglobulin comprising the amino
acid sequence:
(SEQ ID NO: 18)
DIVMTQTPLSLSVTPGQPASISCKASQSLDYEGDSDMNWYLQKPGQPPQL
LIYGASNLESGVPDRFSGSGSGTDFTLKISRVEAEDVGVYYCQQSTEDPR
TEGGGTKVEIKRTVAAPSVFIFPPSDEQLKSGTASVVCLLNNEYPREAKV
QWKVDNALQSGNSQESVTEQDSKDSTYSLSSTLTLSKADYEKHKVYACEV
THQGLSSPVTKSFNRGEC;
and
(SEQ ID NO: 19)
QMQLVQSGPEVKKPGTSVKVSCKASGYTFTDYNVDWVRQARGQRLEWIGD
INPNNGGTIYAQKFQERVTITVDKSTSTAYMELSSLRSEDTAVYYCARNY
RWFGAMDHWGQGTTVTVSSASTKGPSVFPLAPCSRSTSESTAALGCLVKD
YFPEPVTVSWNSGALTSGVHTFPAVLQSSGLYSLSSVVTVPSSSLGTKTY
TCNVDHKPSNTKVDKRVESKYGPPCPPCPAPEFLGGPSVFLEPPKPKDTL
MISRTPEVTCVVVDVSQEDPEVQFNWYVDGVEVHNAKTKPREEQFNSTYR
VVSVLTVLHQDWLNGKEYKCKVSNKGLPSSIEKTISKAKGQPREPQVYTL
PPSQEEMTKNQVSLTCLVKGFYPSDIAVEWESNGQPENNYKTTPPVLDSD
GSFFLYSRLTVDKSRWQEGNVFSCSVMHEALHNHYTQKSLSLSLGK;
or
a light chain immunoglobulin variable domain
comprising the amino acid sequence:
(amino acids 21-131 or SEQ ID NO: 18 (CDRs
underscored))
DIVMTQTPLSLSVTPGQPASISCKASQSLDYEGDSDMNWYLQKPGQPPQL
LIYGASNLESGVPDRFSGSGSGTDFTLKISRVEAEDVGVYYCQQSTEDPR
TFGGGTKVEIK;
and
a heavy chain immunoglobulin variable domain
comprising the amino acid sequence:
(amino acids 1-119 of SEQ ID NO: 19 (CDRs
underscored))
QMQLVQSGPEVKKPGTSVKVSCKASGYTFTDYNVDWVRQARGQRLEWIGD
INPNNGGTIYAQKFQERVTITVDKSTSTAYMELSSLRSEDTAVYYCARNY
RWFGAMDHWGQGTTVTVSS,
or
comprising the CDRs:
CDR-L1:
(SEQ ID NO: 3)
KASQSLDYEGDSDMN;
CDR-L2:
(SEQ ID NO: 4)
GASNLES;
CDR-L3:
(SEQ ID NO: 5)
QQSTEDPRT;
CDR-H1:
(SEQ ID NO: 6)
DYNVD;
CDR-H2:
(SEQ ID NO: 7)
DINPNNGGTIYAQKFQE;
and
CDR-H3:
(SEQ ID NO: 8)
NYRWFGAMDH

Ab6: humanized light chain 45AGX Humanized×[LAG3_H] mAb (LB145.22D2.E1.D1 (VL3)) Kappa (PX) (or the variable domain thereof) and humanized heavy chain 73AHD Humanized×[LAG3_H] mAb (LB145.22D2.E1.D1 VH6 N55D/VL3) IgG4 S228P/Kappa (PX) (or the variable domain thereof); for example comprising:

a light chain immunoglobulin comprising the amino
acid sequence:
(SEQ ID NO: 20)
DIVMTQTPLSLSVTPGQPASISCKASQSLDYEGDSDMNWYLQKPGQPPQL
LIYGASNLESGVPDRFSGSGSGTDFTLKISRVEAEDVGVYYCQQSTEDPR
TFGGGTKVEIKRTVAAPSVFIFPPSDEQLKSGTASVVCLLNNFYPREAKV
QWKVDNALQSGNSQESVTEQDSKDSTYSLSSTLTLSKADYEKHKVYACEV
THQGLSSPVTKSFNRGEC;
and
a heavy chain immunoglobulin comprising the amino
acid sequence:
(SEQ ID NO: 21)
QMQLVQSGPEVKKPGTSVKVSCKASGYTFTDYNVDWVRQARGQRLEWIGD
INPNDGGTIYAQKFQERVTITVDKSTSTAYMELSSLRSEDTAVYYCARNY
RWFGAMDHWGQGTTVTVSSASTKGPSVFPLAPCSRSTSESTAALGCLVKD
YFPEPVTVSWNSGALTSGVHTFPAVLQSSGLYSLSSVVTVPSSSLGTKTY
TCNVDHKPSNTKVDKRVESKYGPPCPPCPAPEFLGGPSVFLEPPKPKDTL
MISRTPEVTCVVVDVSQEDPEVQFNWYVDGVEVHNAKTKPREEQFNSTYR
VVSVLTVLHQDWLNGKEYKCKVSNKGLPSSIEKTISKAKGQPREPQVYTL
PPSQEEMTKNQVSLTCLVKGFYPSDIAVEWESNGQPENNYKTTPPVLDSD
GSFFLYSRLTVDKSRWQEGNVFSCSVMHEALHNHYTQKSLSLSLGK;
or
a light chain immunoglobulin variable domain
comprising the amino acid sequence:
(amino acids 21-131 or SEQ ID NO: 20 (CDRs
underscored))
DIVMTQTPLSLSVTPGQPASISCKASQSLDYEGDSDMNWYLQKPGQPPQL
LIYGASNLESGVPDRFSGSGSGTDFTLKISRVEAEDVGVYYCQQSTEDPR
TFGGGTKVEIK;
and
a heavy chain immunoglobulin variable domain
comprising the amino acid sequence:
(amino acids 1-119 of SEQ ID NO: 21 (CDRs
underscored))
QMQLVQSGPEVKKPGTSVKVSCKASGYTFTDYNVDWVRQARGQRLEWIGD
INPNDGGTIYAQKFQERVTITVDKSTSTAYMELSSLRSEDTAVYYCARNY
RWFGAMDHWGQGTTVTVSS,
or
comprising the CDRs:
CDR-L1:
(SEQ ID NO: 3)
KASQSLDYEGDSDMN;
CDR-L2:
(SEQ ID NO: 4)
GASNLES;
CDR-L3:
(SEQ ID NO: 5)
QQSTEDPRT;
CDR-H1:
(SEQ ID NO: 6)
DYNVD;
CDR-H2:
(SEQ ID NO: 14)
DINPNDGGTIYAQKFQE;
and
CDR-H3:
(SEQ ID NO: 8)
NYRWFGAMDH

Ab7: humanized light chain 45AGX Humanized×[LAG3_H] mAb (LB145.22D2.E1.D1 (VL3)) Kappa (PX) (or the variable domain thereof) and humanized heavy chain 21AHG Humanized×[LAG3_H] mAb (LB145.22D2.E1.D1 VH6 N55S/VL3) IgG4 S228P/Kappa (PX) (or the variable domain thereof); for example comprising:

a light chain immunoglobulin comprising the amino
acid sequence:
(SEQ ID NO: 22)
DIVMTQTPLSLSVTPGQPASISCKASQSLDYEGDSDMNWYLQKPGQPPQL
LIYGASNLESGVPDRFSGSGSGTDFTLKISRVEAEDVGVYYCQQSTEDPR
TEGGGTKVEIKRTVAAPSVFIFPPSDEQLKSGTASVVCLLNNEYPREAKV
QWKVDNALQSGNSQESVTEQDSKDSTYSLSSTLTLSKADYEKHKVYACEV
THQGLSSPVTKSFNRGEC;
and
a heavy chain immunoglobulin comprising the amino
acid sequence:
(SEQ ID NO: 23)
QMQLVQSGPEVKKPGTSVKVSCKASGYTFTDYNVDWVRQARGQRLEWIGD
INPNSGGTIYAQKFQERVTITVDKSTSTAYMELSSLRSEDTAVYYCARNY
RWFGAMDHWGQGTTVTVSSASTKGPSVFPLAPCSRSTSESTAALGCLVKD
YFPEPVTVSWNSGALTSGVHTFPAVLQSSGLYSLSSVVTVPSSSLGTKTY
TCNVDHKPSNTKVDKRVESKYGPPCPPCPAPEFLGGPSVFLFPPKPKDTL
MISRTPEVTCVVVDVSQEDPEVQFNWYVDGVEVHNAKTKPREEQFNSTYR
VVSVLTVLHQDWLNGKEYKCKVSNKGLPSSIEKTISKAKGQPREPQVYTL
PPSQEEMTKNQVSLTCLVKGFYPSDIAVEWESNGQPENNYKTTPPVLDSD
GSFELYSRLTVDKSRWQEGNVESCSVMHEALHNHYTQKSLSLSLGK;
or
a light chain immunoglobulin variable domain
comprising the amino acid sequence:
(amino acids 21-131 or SEQ ID NO: 22 (CDRs
underscored))
DIVMTQTPLSLSVTPGQPASISCKASQSLDYEGDSDMNWYLQKPGQPPQL
LIYGASNLESGVPDRFSGSGSGTDFTLKISRVEAEDVGVYYCQQSTEDPR
TFGGGTKVEIK;
and
a heavy chain immunoglobulin variable domain
comprising the amino acid sequence:
(amino acids 1-119 of SEQ ID NO: 23 (CDRs
underscored))
QMQLVQSGPEVKKPGTSVKVSCKASGYTFTDYNVDWVRQARGQRLEWIGD
INPNSGGTIYAQKFQERVTITVDKSTSTAYMELSSLRSEDTAVYYCARNY
RWFGAMDHWGQGTTVTVSS,
or
comprising the CDRs:
CDR-L1:
(SEQ ID NO: 3)
KASQSLDYEGDSDMN;
CDR-L2:
(SEQ ID NO: 4)
GASNLES;
CDR-L3:
(SEQ ID NO: 5)
QQSTEDPRT;
CDR-H1:
(SEQ ID NO: 6)
DYNVD;
CDR-H2:
(SEQ ID NO: 11)
DINPNSGGTIYAQKFQE;
and
CDR-H3:
(SEQ ID NO: 8)
NYRWFGAMDH

Ab8: humanized light chain 45AGX Humanized×[LAG3_H] mAb (LB145.22D2.E1.D1 (VL3)) Kappa (PX) (or the variable domain thereof) and humanized heavy chain 80AHG Humanized×[LAG3_H] mAb (LB145.22D2.E1.D1 VH6 N55Q/VL3) IgG4 S228P/Kappa (PX) (or the variable domain thereof); for example comprising:

a light chain immunoglobulin comprising the amino
acid sequence:
(SEQ ID NO: 24)
DIVMTQTPLSLSVTPGQPASISCKASQSLDYEGDSDMNWYLQKPGQPPQL
LIYGASNLESGVPDRFSGSGSGTDFTLKISRVEAEDVGVYYCQQSTEDPR
TEGGGTKVEIKRTVAAPSVFIFPPSDEQLKSGTASVVCLLNNEYPREAKV
QWKVDNALQSGNSQESVTEQDSKDSTYSLSSTLTLSKADYEKHKVYACEV
THQGLSSPVTKSFNRGEC;
and
a heavy chain immunoglobulin comprising the amino
acid sequence:
(SEQ ID NO: 25)
QMQLVQSGPEVKKPGTSVKVSCKASGYTFTDYNVDWVRQARGQRLEWIGD
INPNQGGTIYAQKFQERVTITVDKSTSTAYMELSSLRSEDTAVYYCARNY
RWFGAMDHWGQGTTVTVSSASTKGPSVFPLAPCSRSTSESTAALGCLVKD
YFPEPVTVSWNSGALTSGVHTFPAVLQSSGLYSLSSVVTVPSSSLGTKTY
TCNVDHKPSNTKVDKRVESKYGPPCPPCPAPEFLGGPSVFLFPPKPKDTL
MISRTPEVTCVVVDVSQEDPEVQFNWYVDGVEVHNAKTKPREEQFNSTYR
VVSVLTVLHQDWLNGKEYKCKVSNKGLPSSIEKTISKAKGQPREPQVYTL
PPSQEEMTKNQVSLTCLVKGFYPSDIAVEWESNGQPENNYKTTPPVLDSD
GSFFLYSRLTVDKSRWQEGNVFSCSVMHEALHNHYTQKSLSLSLGK;
or
a light chain immunoglobulin variable domain
comprising the amino acid sequence:
DIVMTQTPLSLSVTPGQPASISCKASQSLDYEGDSDMNWYLQKPGQPPQL
LIYGASNLESGVPDRFSGSGSGTDFTLKISRVEAEDVGVYYCQQSTEDPR
TFGGGTKVEIK (amino acids 21-131 or SEQ ID NO: 24
(CDRs underscored));
and
a heavy chain immunoglobulin variable domain
comprising the amino acid sequence:
QMQLVQSGPEVKKPGTSVKVSCKASGYTFTDYNVDWVRQARGQRLEWIGD
INPNQGGTIYAQKFQERVTITVDKSTSTAYMELSSLRSEDTAVYYCARNY
RWFGAMDHWGQGTTVTVSS (amino acids 1-119 of SEQ ID
NO: 25 (CDRs underscored)),
or
comprising the CDRs:
CDR-L1:
(SEQ ID NO: 3)
KASQSLDYEGDSDMN;
CDR-L2:
(SEQ ID NO: 4)
GASNLES;
CDR-L3:
(SEQ ID NO: 5)
QQSTEDPRT;
CDR-H1:
(SEQ ID NO: 6)
DYNVD;
CDR-H2:
(SEQ ID NO: 17)
DINPNQGGTIYAQKFQE;
and
CDR-H3:
(SEQ ID NO: 8)
NYRWFGAMDH

or
Ab9: humanized light chain 45AGX Humanized×[LAG3_H] mAb (LB145.22D2.E1.D1 (VL3)) Kappa (PX) (or the variable domain thereof) and humanized heavy chain 72AHD Humanized×[LAG3_H] mAb (LB145.22D2.E1.D1 VH6 N55G/VL3) IgG4 S228P/Kappa (PX)) (or the variable domain thereof); for example comprising:

a light chain immunoglobulin comprising the amino
acid sequence:
(SEQ ID NO: 26)
DIVMTQTPLSLSVTPGQPASISCKASQSLDYEGDSDMNWYLQKPGQPPQ
LLIYGASNLESGVPDRFSGSGSGTDFTLKISRVEAEDVGVYYCQQSTED
PRTEGGGTKVEIKRTVAAPSVFIFPPSDEQLKSGTASVVCLLNNEYPRE
AKVQWKVDNALQSGNSQESVTEQDSKDSTYSLSSTLTLSKADYEKHKVY
ACEVTHQGLSSPVTKSFNRGEC;
and
a heavy chain immunoglobulin comprising the amino
acid sequence:
(SEQ ID NO: 27)
QMQLVQSGPEVKKPGTSVKVSCKASGYTFTDYNVDWVRQARGQRLEWIG
DINPNGGGTIYAQKFQERVTITVDKSTSTAYMELSSLRSEDTAVYYCAR
NYRWFGAMDHWGQGTTVTVSSASTKGPSVFPLAPCSRSTSESTAALGCL
VKDYFPEPVTVSWNSGALTSGVHTFPAVLQSSGLYSLSSVVTVPSSSLG
TKTYTCNVDHKPSNTKVDKRVESKYGPPCPPCPAPEFLGGPSVFLFPPK
PKDTLMISRTPEVTCVVVDVSQEDPEVQFNWYVDGVEVHNAKTKPREEQ
FNSTYRVVSVLTVLHQDWLNGKEYKCKVSNKGLPSSIEKTISKAKGQPR
EPQVYTLPPSQEEMTKNQVSLTCLVKGFYPSDIAVEWESNGQPENNYKT
TPPVLDSDGSFFLYSRLTVDKSRWQEGNVFSCSVMHEALHNHYTQKSLS
LSLGK;
or
a light chain immunoglobulin variable domain
comprising the amino acid sequence:
DIVMTQTPLSLSVTPGQPASISCKASQSLDYEGDSDMNWYLQKPGQPPQ
LLIYGASNLESGVPDRFSGSGSGTDFTLKISRVEAEDVGVYYCQQSTED
PRTFGGGTKVEIK (amino acids 21-131 or SEQ ID NO:
26 (CDRs underscored));
and
a heavy chain immunoglobulin variable domain
comprising the amino acid sequence:
QMQLVQSGPEVKKPGTSVKVSCKASGYTFTDYNVDWVRQARGQRLEWIG
DINPNGGGTIYAQKFQERVTITVDKSTSTAYMELSSLRSEDTAVYYCAR
NYRWFGAMDHWGQGTTVTVSS (amino acids 1-119 of SEQ
ID NO: 27 (CDRs underscored)), or comprising the
CDRs:
CDR-L1:
(SEQ ID NO: 3)
KASQSLDYEGDSDMN;
CDR-L2:
(SEQ ID NO: 4)
GASNLES;
CDR-L3:
(SEQ ID NO: 5)
QQSTEDPRT;
CDR-H1:
(SEQ ID NO: 6)
DYNVD;
CDR-H2:
(SEQ ID NO: 28)
DINPNGGGTIYAQKFQE;
and
CDR-H3:
(SEQ ID NO: 8)
NYRWFGAMDH

In an embodiment of the invention, the CDR-H2 of any anti-LAG3 antibody or antigen-binding fragment thereof of the present invention comprises the amino acid sequence: DINPNX₁GGTIYX₂QKFX₃X₄ (SEQ ID NO: 29)

wherein,

### X₁=D,N,S or Q

### X₂=A or S

### X₃=Q or K

### X₄=E or G

Other anti-LAG3 antibodies and antigen-binding fragments are described below.

In an embodiment of the invention, the anti-LAG3 antibody or antigen-binding fragment thereof comprises the following CDRs:

CDR-H1:
(SEQ ID NO: 30)
DYYWN;
CDR-H2:
(SEQ ID NO: 31)
EINHNGNTNSNPSLKS;
CDR-H3:
(SEQ ID NO: 32)
GYSDYEYNWFDP;
CDR-L1:
(SEQ ID NO: 33)
RASQSISSYLA;
CDR-L2:
(SEQ ID NO: 34)
DASNRAT;
and
CDR-L3:
(SEQ ID NO: 35)
QQRSNWPLT

In an embodiment of the invention, the anti-LAG3 antibody or antigen-binding fragment thereof comprises the following CDRs:

CDR-H1:
(SEQ ID NO: 36)
SYGMH;
CDR-H2:
(SEQ ID NO: 37)
VIWYDGSNKYYADSVKG;
CDR-H3:
(SEQ ID NO: 38)
EWAVASWDYGMDV;
CDR-L1:
(SEQ ID NO: 39)
RASQSVSSSYLA;
CDR-L2:
(SEQ ID NO: 40)
GASSRAT;
and
CDR-L3:
(SEQ ID NO: 41)
QQYGSSPFT

In an embodiment of the invention, the anti-LAG3 antibody or antigen-binding fragment thereof comprises the following CDRs:

CDR-H1:
(SEQ ID NO: 42)
DYGMS;
CDR-H2:
(SEQ ID NO: 43)
GINWNGGSTYYADSVKG;
CDR-H3:
(SEQ ID NO: 44)
PVGVV;
CDR-L1:
(SEQ ID NO: 45)
RASQGIRSALA;
CDR-L2:
(SEQ ID NO: 46)
DASSLES;
and
CDR-L3:
(SEQ ID NO: 47)
QQFNSYPYT

In an embodiment of the invention, the anti-LAG3 antibody or antigen-binding fragment thereof comprises the following CDRs:

CDR-H1:
(SEQ ID NO: 48)
GYYWS;
CDR-H2:
(SEQ ID NO: 49)
EINHRGNTNCNPSLKS;
CDR-H3:
(SEQ ID NO: 50)
GYDILTGYYEDS;
CDR-L1:
(SEQ ID NO: 51)
RASQSVSSYLA;
CDR-L2:
(SEQ ID NO: 52)
NASNRAT;
and
CDR-L3:
(SEQ ID NO: 53)
QQRSNWPLT

In an embodiment of the invention, the anti-LAG3 antibody or antigen-binding fragment thereof comprises the following CDRs:

CDR-H1:
(SEQ ID NO: 54)
EVSMH;
CDR-H2:
(SEQ ID NO: 55)
GFDPEDGETIYAQKFQG;
CDR-H3:
(SEQ ID NO: 56)
AFVVVVAASDY;
CDR-L1:
(SEQ ID NO: 57)
RASQSVSSYLA;
CDR-L2:
(SEQ ID NO: 58)
DASNRAT;
and
CDR-L3:
(SEQ ID NO: 59)
QQRSNWPWT

In an embodiment of the invention, the anti-LAG3 antibody or antigen-binding fragment thereof comprises the following CDRs:

CDR-H1:
(SEQ ID NO: 60)
SYGMH;
CDR-H2:
(SEQ ID NO: 61)
VIWYDGSNKYYADSVKG;
CDR-H3:
(SEQ ID NO: 62)
DPHCSSTNCYLFDY;
CDR-L1:
(SEQ ID NO: 63)
RASQSVSSYLA;
CDR-L2:
(SEQ ID NO: 64)
DASNRAT;
and
CDR-L3:
(SEQ ID NO: 65)
QQRSNWPIT

In an embodiment of the invention, the anti-LAG3 antibody or antigen-binding fragment thereof comprises the following CDRs:

CDR-H1:
(SEQ ID NO: 66)
GFSLTAYG;
CDR-H2:
(SEQ ID NO: 67)
IWDDGST;
CDR-H3:
(SEQ ID NO: 68)
AREGDVAFDY;
CDR-L1:
(SEQ ID NO: 69)
QSLLNGSNQKNY;
CDR-L2:
(SEQ ID NO: 70)
FAS;
and
CDR-L3:
(SEQ ID NO: 71)
LQHFGTPPT

In an embodiment of the invention, the anti-LAG3 antibody or antigen-binding fragment thereof comprises the following CDRs:

CDR-H1:
(SEQ ID NO: 72)
AYGVN
CDR-H2:
(SEQ ID NO: 73)
MIWDDGSTDYDSALKS
CDR-H3:
(SEQ ID NO: 74)
EGDVAFDY
CDR-L1:
(SEQ ID NO: 75)
KSSQSLLNPSNQKNYLA
CDR-L2:
(SEQ ID NO: 76)
FASTRDS
CDR-L3:
(SEQ ID NO: 77)
LQHFGTPPT
See WO2010/19570 and WO2014/140180.

In an embodiment of the invention, the anti-LAG3 antibody or antigen-binding fragment thereof comprises immunoglobulin chains comprising the amino acid sequences: Light chain:

(SEQ ID NO: 84)
EIVLTQSPGTLSLSPGERATLSCRASQSVSSSYLAWYQQKPGQAPRLLIY
GASSRATGIPDRFSGSGSGTDFTLTISRLEPEDFAVYYCQQYGSSPFTFG
PGTKVDIK
Heavy chain:
(SEQ ID NO: 85)
QVQLVESGGGVVQPGRSLRLSCAASGFTESSYGMHWVRQAPGKGLEWVAV
IWYDGSNKYYADSVKGRETISRDNSKNTLYLQMNSLRAEDTAVYYCAREW
AVASWDYGMDVWGQGTTVTVSS

In an embodiment of the invention, the anti-LAG3 antibody or antigen-binding fragment thereof is 17B4 or 4F4. Baixeras et al. J. Exp. Med. 176:327-337 (1992).

In an embodiment of the invention, the test substance is IMP321 (or any recombinant soluble human LAG3-Ig fusion protein). In an embodiment of the invention, the test substance is the whole LAG3 protein, a soluble polypeptide fragment thereof including at least one of the four immunoglobulin extracellular domains, e.g., the soluble part of LAG-3 including the extracellular region stretching from the amino acid 23 to the amino acid 448 of LAG3, a fragment of LAG3 consisting of substantially all of the first and second domains, a fragment of LAG3 including substantially all of the first and second domains or all of the four domains, a mutant form of soluble LAG3 or a fragment thereof comprising the D1 and D2 extracellular domains and having:

a substitution of an amino acid at one of the following positions:
position 73 where ARG is substituted with GLU,
position 75 where ARG is substituted with ALA or GLU,
position 76 where ARG is substituted with GLU,
or a combination of two or more of those substitutions; and/or
a substitution of an amino acid at one of the following positions:
position 30 where ASP is substituted with ALA,
position 56 where HIS is substituted with ALA,
position 77 where TYR is substituted with PHE,
position 88 where ARG is substituted with ALA,
position 103 where ARG is substituted with ALA,
position 109 where ASP is substituted with GLU,
position 115 where ARG is substituted with ALA,
or a deletion of the region comprised between the position 54 and the position 66,
or a combination of two or more of those substitutions. See human LAG3 sequence below.

### LAG3

The term “LAG3”, with respect to the polypeptide or polynucleotide, e.g., to which antibodies and antigen-binding fragments of the present invention bind, includes human and cynomolgous monkey, e.g., Macaca fascicularis or Macaca mulatta LAG3 as well as fragments thereof such as the mature fragment thereof lacking the signal peptide.

In an embodiment of the invention, the amino acid sequence of human LAG3 comprises:

| (SEQ ID NO: 78) | |
|---|---|
| MWEAQFLGLL FLQPLWVAPV KP | |
| LQPGAEVPVV WAQEGAPAQL PCSPTIPLQD LSLLRRAGVT WQHQPDSGPP AAAPGHPLAP | 60 |
| GPHPAAPSSW GPRPRRYTVL SVGPGGLRSG RLPLQPRVQL DERGRQRGDF SLWLRPARRA | 120 |
| DAGEYRAAVH LRDRALSCRL RLRLGQASMT ASPPGSLRAS DWVILNCSFS RPDRPASVHW | 180 |
| FRNRGQGRVP VRESPHHHLA ESFLFLPQVS PMDSGPWGCI LTYRDGFNVS IMYNLTVLGL | 240 |
| EPPTPLTVYA GAGSRVGLPC RLPAGVGTRS FLTAKWTPPG GGPDLLVTGD NGDFTLRLED | 300 |
| VSQAQAGTYT CHIHLQEQQL NATVTLAIIT VTPKSFGSPG SLGKLLCEVT PVSGQERFVW | 360 |
| SSLDTPSQRS FSGPWLEAQE AQLLSQPWQC QLYQGERLLG AAVYFTELSS PGAQRSGRAP | 420 |
| GALPAGHLLL FLILGVLSLL LLVTGAFGFH LWRRQWRPRR FSALEQGIHP PQAQSKIEEL | 480 |
| EQEPEPEPEP EPEPEPEPEP EQL; | 503 |

signal sequence underscored and bold; see also Uniprot accession no. P18627.

In an embodiment of the invention, the amino acid sequence of cynomolgous monkey LAG3 comprises:

(SEQ ID NO: 79)
MWEAQFLGLL FLQPLWVAPV KP
PQPGAEIS VVWAQEGAPA QLPCSPTIPL QDLSLLRRAG
VTWQHQPDSG PPAXAPGHPP VPGHRPAAPY SWGPRPRRYT
VLSVGPGGLR SGRLPLQPRV QLDERGRQRG DFSLWLRPAR
RADAGEYRAT VHLRDRALSC RLRLRVGQAS MTASPPGSLR
TSDWVILNCS FSRPDRPASV HWFRSRGQGR VPVQGSPHHH
LAESFLFLPH VGPMDSGLWG CILTYRDGFN VSIMYNLTVL
GLEPATPLTV YAGAGSRVEL PCRLPPAVGT QSFLTAKWAP
PGGGPDLLVA GDNGDFTLRL EDVSQAQAGT YICHIRLQGQ
QLNATVTLAI ITVTPKSFGS PGSLGKLLCE VTPASGQEHF
VWSPLNTPSQ RSFSGPWLEA QEAQLLSQPW QCQLHQGERL
LGAAVYFTEL SSPGAQRSGR APGALRAGHL PLFLILGVLF
LLLLVTGAFG FHLWRRQWRP RRFSALEQGI HPPQAQSKIE
ELEQEPELEP EPELERELGP EPEPGPEPEP EQL;

signal sequence underscored and bold; see also NCBI reference number XP_005570011.1

### T-Cells and Antigen-Presenting Cells

The present invention encompasses methods of using cells and compositions that are useful, for example, in connection with the assays discussed herein. For example, the present invention provides T-cells that are capable of an optimal T-cell receptor stimulation response. It has been surprisingly discovered that T-cells exhibit such optimal stimulation response if the levels of LAG3 and CD3 are at a ratio of about 2/1 (LAG3/CD3) or less (e.g., about 0.1, 0.2, 0.6, 0.8, 1, 1.1 or 1.6 or about 0.1-1.6; 0.1-2.0, 0.2-1.6, 0.6-1.6, 0.8-1.6, 1.0-1.6, 1.1-1.6, 0.2-2.0, 0.2-1.6, 0.2-1.1, 0.2-1.0, 0.2-0.8 or 0.2-0.6). As used herein, the term “ratio of 2/1 or lower” (or the like), with respect to the expression of LAG3 and CD3 on a T-cell, does not include embodiments wherein LAG3 or CD3, in the ratio, is zero. In an embodiment of the invention, the ratio of LAG3 to CD3 (LAG3/CD3) refers to:

$$\frac{M F I T \text{-} cell ( anti \text{-} L A G 3 ) / M F I T \text{-} cell ( L A G 3 control )}{M F I T \text{-} cell ( anti \text{-} CD 3 ) / M F I T \text{-} cell ( CD 3 control )} ;$$

wherein:

MFI T-cell (anti-LAG3) is the mean fluorescent intensity observed in FACS analysis of the T-cell stained with anti-LAG3 such as Ab6 (IgG4/x) or with an antibody comprising the immunoglobulin chains of SEQ ID NOs: 84 and 85 (IgG1/x) labeled with DyLight650,

MFI T-cell (LAG3 control) is the mean fluorescent intensity observed in FACS analysis of the T-cell stained with a control antibody (e.g., an control antibody of the anti-LAG3 antibody used above), such as trastuzumab-DyLight 650;

MFI T-cell (anti-CD3) is the mean fluorescent intensity observed in FACS analysis of the T-cell stained with anti-CD3 such as maHuCD3 pacblue; and

MFI T-cell (CD3 control) is the mean fluorescent intensity observed in FACS analysis of the T-cell stained with a control antibody (e.g., a control antibody of the anti-CD3 antibody used above) such as mIgG1-pacblue.

mIgG1-pacblue is a mouse IgG1/κ MOPC-21 antibody of unknown specificity from mouse myeloma that is commercially available, for example, from BD Pharmigen.

maHuCD3 pacblue is a mouse anti-human CD3E antibody Clone SP34-2 (IgG1/2) that is commercially available, for example, from BD Pharmigen. Clone SP34-2 is a mouse IgG1 isotype monoclonal antibody, descendant of SP34 (mouse IgG3), with the same specificity and reactivity pattern as the parent clone. Alarcon et al. EMBO J. 1991 April; 10(4):903-12; Carter et al. Cytometry. 1999; 37(1):41-50; Sancho et al. J Biol Chem. 1992; 267(11):7871-7879; Schlossman S F, Boumsell L, Gilks W, et al., ed. Leucocyte Typing V. New York: Oxford University Press; 1995; Wilson et al. J Immunol Methods. 1995; 178(2):195-200; Salmeron et al., (1991) J. Immunol. 147: 3047.

Trastuzumab is a commercially available anti-HER2 monoclonal antibody that is well known in the art. See e.g., Hudis, N Engl J Med 357:39-51 (2007).

In an embodiment of the invention, the T-cells express PD-1, e.g., human PD-1 and/or the antigen-presenting cell expresses PD-L1, e.g., human PD-L1

In an embodiment of the invention, the anti-human CD3 used for staining to determine the ratio set forth above is HIT3a, UCHT1, OKT3, SK7, APA1/1 or SP34-2, and/or the anti-LAG3 used for staining to determine the ratio set forth above is any of the anti-LAG3 antibodies or antigen-binding fragments thereof that are set forth herein; and/or the control antibodies are antibodies or antigen-binding fragments which are known not to exhibit significant binding to CD3 or LAG3, respectively.

Pacific Blue (pacblue) fluorescent dye is commercially available for example, from BD Pharmigen. The Pacific Blue label is based on the 6,8-difluoro-7-hydroxycoumarin fluorophore and is strongly fluorescent, even at neutral pH. Pacific Blue has a maximum absorption of 416 nm and maximum emission of 451 nm. The Pacific Blue succinimidyl ester is:

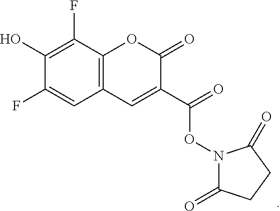 [SMILES: O=C(ON1C(=O)CCC1=O)c1cc2cc(F)c(O)c(F)c2oc1=O]

DyLight 650 fluorescent dye

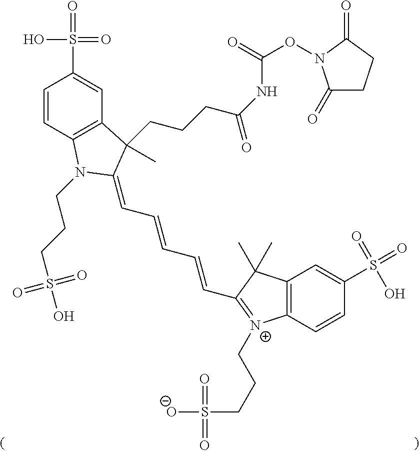 [SMILES: CC1(C)C(/C=C/C=C/C=C2/N(CCCS(=O)(=O)O)c3ccc(S(=O)(=O)O)cc3C2(C)CCCC(=O)NC(=O)ON2C(=O)CCC2=O)=[N+](CCCS(=O)(=O)[O-])c2ccc(S(=O)(=O)O)cc21]

is commercially available, for example, from BD Pharmigen. DyLight 650 can be excited at 652 nm and emits at 672 nm (molar extinction coefficient 250,000 M⁻¹ cm⁻¹). DyLight and its uses are commonly known in the art. See e.g., Liu et al. PLoS One. 2012; 7(6):e40003; Maawy et al., J. Biomed. Opt. 2013 18(12):126016; and Maawy et al. PLoS One. 2014 9(5):e97965.

In an embodiment of the invention, the staining intensity of the antibody-labeled T-cells are calculated by:
incubating T-cells with a mixture of anti-CD3 and/or anti-LAG3 which are labeled with different fluorescent labels;
incubating T-cells with a mixture of control antibodies, e.g., of the same isotype as anti-CD3 and an antibody of the same isotype as anti-LAG3, which are labeled with different fluorescent labels, for example, for about 30 minutes;
optionally, washing (e.g., with phosphate buffer saline and bovine serum albumin) and fixing the T-cells (e.g., with paraformaldehyde), for example, at 4° C.; and
quantitating fluorescence of the antibody labeled cells, e.g., in a FACS apparatus, for example, by activating the fluorescent labels at their excitation wavelengths and determining fluorescent intensities at the label fluorescent emission wavelengths.

In an embodiment of the invention, the ratio of 2/1 (LAG3/CD3) refers to the ratio of the copy numbers of LAG3 and CD3 on a T-cell.

Human and cynomolgous monkey T-cells that are suitable for use in the assays of the present invention include those which express LAG3 and CD3 at a ratio of about 2/1 (LAG3/CD3) or less (e.g., about 1/1). In an embodiment of the invention, a human T-cell is Jurkat; CCRF-CEM; HPB-ALL; HPB-MLT; HD-Mar-2; TALL-I; MOLT-16, MAT; H9; ED-S; ATL-35T; MJ; Kit225; HuT 102; HuT 78 or HH. Such T-cells can be modified by introduction of a polynucleotide encoding LAG3 into the cells such that the ratio of LAG3 to CD3 is about 2/1 or less.

Antigen-presenting cells (APCs) are cells (e.g., human, mouse or non-human primate such as monkey, e.g., cynomolgous monkey) that express human or non-human primate (e.g., monkey, such as cynomolgous monkey) major histocompatibility complex (MHC) class II and present antigen to which a T-cell binds. In an embodiment of the invention, the APC is a Raji B-cell lymphoma cell. In an embodiment of the invention, the APC is a Daudi cell, a JY cell, a melanoma cell, or an L-cell that overexpresses HLA-DR or B7. In an embodiment of the invention, the APC is a melanoma cell such as A375; Mel1, Meljuso; WM983A; WM983B; SLM8; HM11 or WAC. In an embodiment of the invention, the APC is a B-cell such as SR, Ramos, Pfieffer, or RPMI 8226. Thus, the present invention includes compositions comprising a T-cell (e.g., a Jurkat cell) expressing LAG3 and CD3 at a ratio of about 2/1 (LAG3/CD3) or less and an APC (e.g., a Raji cell).

The present invention provides compositions comprising a T-cell of the present invention that expresses LAG3 and CD3 are at a ratio of about 2/1 (LAG3/CD3) or less (e.g., a Jurkat cell) and a T-cell activating agent such as Staphylococcal enterotoxin B (SEB) (e.g., but not SEB if the T-cell is a Jurkat cell), Staphylococcal enterotoxin A (SEA), Staphylococcal enterotoxin D (SED) or Staphylococcal enterotoxin E (SEE).

The present invention provides compositions including a T-cell of the present invention (e.g., a Jurkat cell) that express LAG3 and CD3 are at a ratio of about 2/1 (LAG3/CD3) or less (e.g., 1.5/1, 1/1, 1/2) and a LAG3 inhibitor or agonist, such as anti-LAG3 antibody or antigen-binding fragment thereof such as any of those specifically set forth herein (e.g., Ab1, Ab2, Ab3, Ab4, Ab5, Ab6, Ab7, Ab8 or Ab9) or a LAG3 polypeptide (e.g., a LAG3-Ig fusion).

Compositions comprising a T-cell of the present invention (e.g., a Jurkat cell) expressing LAG3 and CD3 at a ratio of about 2/1 or less in the presence of an antigen-presenting cell (e.g., a Raji cell), e.g., wherein the ratio of T-cells to antigen-presenting cells is about 1/1, 2/1, 4/1, 8/1, 16/1 or 32/1 (e.g., 1-32/1), are part of the present invention. In an embodiment of the invention, a composition of T-cells and antigen-presenting cells comprises T-cells at about 8×10⁶ cells/ml and antigen-presenting cells at about 2×10⁵ cells/ml. For example, in an embodiment of the invention, each assay is conducted with about 100,000 T cells and about 25,000 APCs (e.g., cells per well). The present invention provides compositions comprising about 100,000 T-cells expressing LAG3 and CD3 at a ratio of about 2 or less and about 25,000 APCs. The present invention also includes compositions wherein the T-cell and the APC are in separate vessels or containers. The T-cell and the APC in separate containers may be combined into a single vessel, e.g., for use in a method described herein.

In an embodiment of the invention, T-cells that express LAG3 and CD3 at a ratio of about 2/1 (LAG3/CD3) or lower (e.g., 1.5/1, 1/1, 1/2) are generated by introducing a polynucleotide encoding LAG3 into the T-cell. In an embodiment of the invention, a LAG3 polynucleotide is introduced (e.g., transduced) into the T-cell by introducing a polynucleotide encoding LAG3 into the cell in a vector, such as a retroviral vector, that includes the polynucleotide encoding LAG3, e.g., having a leader sequence such as a VEGF leader sequence. In an embodiment of the invention, the vector is a lentiviral vector. In an embodiment of the invention, the LAG3 having a VEGF leader sequence (underscored) comprises the amino acid sequence:

(SEQ ID NO: 81)
MNFLLSWVHWSLALLLYLHHAKWSQALQPGAEVPVVWAQEGAPAQLPCSP
TIPLQDLSLLRRAGVTWQHQPDSGPPAAAPGHPLAPGPHPAAPSSWGPRP
RRYTVLSVGPGGLRSGRLPLQPRVQLDERGRQRGDFSLWLRPARRADAGE
YRAAVHLRDRALSCRLRLRLGQASMTASPPGSLRASDWVILNCSFSRPDR
PASVHWERNRGQGRVPVRESPHHHLAESELFLPQVSPMDSGPWGCILTYR
DGENVSIMYNLTVLGLEPPTPLTVYAGAGSRVGLPCRLPAGVGTRSELTA
KWTPPGGGPDLLVTGDNGDFTLRLEDVSQAQAGTYTCHIHLQEQQLNATV
TLAIITVTPKSEGSPGSLGKLLCEVTPVSGQERFVWSSLDTPSQRSFSGP
WLEAQEAQLLSQPWQCQLYQGERLLGAAVYFTELSSPGAQRSGRAPGALP
AGHLLLFLILGVLSLLLLVTGAFGFHLWRRQWRPRRESALEQGTHPPQAQ
SKIEELEQEPEPEPEPEPEPEPEPEPEQL.

In an embodiment of the invention, the LAG3 having a VEGF leader sequence is encoded by a polynucleotide comprising the nucleotide sequence:

(SEQ ID NO: 82)
ATGAACTTTCTGCTGAGCTGGGTGCACTGGTCCCTGGCCCTGCTGCTGT
ACCTGCACCACGCTAAGTGGTCCCAGGCTCTGCAGCCTGGCGCTGAAGT
GCCTGTCGTGTGGGCTCAGGAAGGCGCTCCTGCCCAGCTGCCTTGCAGC
CCTACAATCCCACTGCAGGACCTGAGCCTGCTGAGAAGGGCTGGCGTGA
CATGGCAGCACCAGCCTGATTCTGGACCTCCTGCTGCTGCTCCTGGACA
CCCACTGGCTCCAGGACCTCACCCAGCTGCTCCAAGCTCTTGGGGCCCC
AGACCTAGAAGATACACCGTGCTGTCTGTGGGCCCTGGCGGCCTGAGAT
CTGGTAGACTGCCTCTGCAGCCAAGAGTGCAGCTGGACGAGAGAGGCAG
ACAGAGGGGCGACTTCAGCCTGTGGCTGAGGCCAGCTAGAAGGGCCGAT
GCCGGCGAGTACAGAGCCGCCGTGCATCTGAGAGACAGAGCCCTGAGCT
GCAGACTGAGACTGAGGCTGGGCCAGGCCAGCATGACAGCTAGCCCTCC
AGGCAGCCTGAGAGCCAGCGACTGGGTCATCCTGAACTGCAGCTTCAGC
AGACCCGACAGACCCGCCAGCGTGCACTGGTTCAGAAACAGAGGCCAGG
GCAGAGTGCCCGTGCGCGAGTCTCCTCACCACCACCTGGCCGAGAGCTT
TCTGTTCCTGCCACAGGTGTCCCCCATGGACTCTGGCCCTTGGGGCTGC
ATCCTGACCTACAGGGACGGCTTCAACGTGTCCATCATGTACAACCTGA
CCGTGCTGGGCCTGGAACCCCCTACCCCTCTGACAGTGTACGCTGGCGC
TGGCTCTAGAGTGGGCCTGCCTTGTAGACTGCCAGCCGGCGTGGGCACC
AGAAGCTTTCTGACCGCCAAGTGGACACCTCCCGGCGGAGGACCTGATC
TGCTGGTCACCGGCGACAACGGCGACTTCACACTGAGACTGGAAGATGT
GTCCCAGGCCCAGGCCGGCACCTACACCTGTCACATCCATCTGCAGGAA
CAGCAGCTGAACGCCACCGTGACCCTGGCCATCATCACCGTGACACCCA
AGAGCTTCGGCAGCCCTGGCTCTCTGGGCAAGCTGCTGTGTGAAGTGAC
CCCCGTGTCCGGCCAGGAAAGATTCGTCTGGTCCAGCCTGGACACACCC
AGCCAGAGAAGCTTCAGCGGCCCTTGGCTGGAAGCCCAGGAAGCACAGC
TGCTGAGCCAGCCTTGGCAGTGCCAGCTGTACCAGGGCGAGAGACTGCT
GGGCGCTGCCGTGTACTTCACCGAGCTGAGTAGCCCTGGCGCCCAGAGA
AGTGGTAGAGCACCTGGTGCCCTGCCTGCCGGCCATCTGCTGCTGTTTC
TGATCCTGGGCGTGCTGTCCCTGCTCCTGCTCGTGACAGGCGCTTTCGG
CTTCCACCTGTGGCGGAGACAGTGGCGGCCTAGAAGATTCAGCGCCCTG
GAACAGGGCATCCACCCTCCACAGGCCCAGAGCAAGATCGAGGAACTGG
AACAGGAACCCGAGCCCGAGCCTGAGCCCGAACCAGAACCTGAGCCTGA
ACCTGAGCCAGAGCAGCTGTGA.

In an embodiment of the invention, the polynucleotide encoding the LAG3 polypeptide is operably linked to a Kozak sequence, e.g., GCCGCCACC (SEQ ID NO: 83).

The present invention provides methods comprising use of T-cells comprising a vector-borne LAG3 polynucleotide, e.g., operably linked to a promoter, as well as such cells themselves. In an embodiment of the invention, when making such T-cells, after introduction (e.g., transduction) of LAG3, the cells are screened and cells that express LAG3 and CD3 at a ratio of about 2/1 (LAG3/CD3) or lower (e.g., about 0.2, 0.6, 0.8, 1, 1.1 or 1.6 or about 0.2-1.6) are selected. For example, in an embodiment of the invention, T-cells are screened by staining the cells with detectable agents that bind LAG3 and CD3 and cells expressing the appropriate ratio of LAG3 and CD3 are selected. For example, in an embodiment of the invention, transduced or transformed T-cells are screened and cells expressing the LAG3 and CD3 at a ratio of about 2/1 or lower (e.g., about 0.2, 0.6, 0.8, 1, 1.1 or 1.6 or about 0.2-1.6) are selected by fluorescence-activated cell sorting (FACS). For example, fluorescent agents (e.g., antibodies labeled with a fluorescent moiety) that bind LAG3 or CD3, which can be employed for screening cells. If the detectable agents are antibodies or antigen-binding fragments or other molecules that fluoresce and bind to LAG3 and CD3, then fluorescence of transduced or transformed T-cells bound to the detectable agents can be evaluated and cells expressing fluorescence a ratio that indicates a LAG3 to CD3 expression ratio of about 2/1 or lower (e.g., about 0.2, 0.6, 0.8, 1, 1.1 or 1.6 or about 0.2-1.6) can be selected, for example, selected and sorted from other cells with which they are mixed.

The present invention also provides a method for making such a composition comprising T-cells of the present invention (e.g., Jurkat cells expressing LAG3 and CD3 at a ratio of about 2/1 or lower) in the presence of antigen-presenting cells (e.g., Raji cells). The present invention provides a method for making a composition comprising a T-cell that expresses LAG3 and CD3 at a ratio of about 2/1 or lower (e.g., about 0.2, 0.6, 0.8, 1, 1.1 or 1.6 or about 0.2-1.6) and an antigen-presenting cell comprising combining such T-cells and such APCs into a single composition. In an embodiment of the invention, the method comprises the steps:

(i) introducing a polynucleotide encoding LAG3 into T-cells;
(ii) selecting T-cells comprising the LAG3 polynucleotide that express LAG3 and CD3 at a ratio of about 2/1 (LAG3/CD3) or lower (e.g., about 0.2, 0.6, 0.8, 1, 1.1 or 1.6 or about 0.2-1.6); and
(iii) combining the T-cells expressing LAG3 and CD3 at a ratio of about 2/1 or lower with antigen-presenting cells into a single composition.

In an embodiment of the invention, the method comprises the steps:

(i) incubating antigen-presenting cells (APCs) with a T-cell activating agent (e.g., a staphylococcal enterotoxin);
(ii) incubating T-cells expressing LAG3 and CD3 at a ratio of about 2/1 or lower with a LAG3 inhibitor or agonist; and
(iii) combining the compositions from steps (i) and (ii) into a single composition.

In an embodiment of the invention, the method comprises the steps:

(i) introducing a polynucleotide encoding LAG3 into T-cells;
(ii) selecting T-cells comprising the LAG3 polynucleotide that express LAG3 and CD3 at a ratio of about 2 or lower (e.g., about 0.2, 0.6, 0.8, 1, 1.1 or 1.6 or about 0.2-1.6); and
(iii) incubating the T-cells expressing LAG3 and CD3 at a ratio of about 2/1 or lower with a LAG3 inhibitor or agonist;
(iv) incubating antigen-presenting cells (APCs) with a T-cell activating agent (e.g., a staphylococcal enterotoxin);
(v) combining the T-cells from step (iii) and the APCs from step (iv) into a single composition.

A composition that is the product of any of such methods is a part of the present invention.

### Assay

The present invention provides methods for determining whether or to what extent a test substance (e.g., a LAG3 inhibitor or agonist) stimulates T-cells, wherein the methods make use of the superior antigenic response exhibited by the T-cells of the present invention which express LAG3 and CD3 at a ratio of about 2/1 (LAG3/CD3) or less (e.g., about 0.2, 0.6, 0.8, 1, 1.1 or 1.6 or about 0.2-1.6).

A test substance is determined to immunostimulate T-cells if, for example, the T-cells, when in contact with the test substance, exhibit an increased state of activation, e.g., increased secretion of cytokines (e.g., IL-2) or increased intracellular T-cell activation pathway induction, such as by activation of expression from the NFAT Response Element (NFAT-RE). Test substances may be found to be LAG3 inhibitors or LAG3 agonists. Specifically, a test substance may cause increased T-cell activation or may cause decreased T-cell activation.

These assays can also be used to determine whether or to what extent the combination of anti-LAG3 and another modulator of an immunomodulatory receptor (IMR), such as PD-1, stimulates T-cells. In such an embodiment, the T-cell expresses LAG3 and the other immunomodulatory receptor, e.g., PD-1 and the antigen-presenting cell expresses the ligand for the other immunomodulatory receptor, e.g., PD-L1 if the T-cell expresses PD-1. In these assays the T-cells and APCs are contacted with anti-LAG3 and the modulator of the IMR. For example, if the other IMR is PD-1, then the modulator of the other IMR can be pembrolizumab or nivolumab or another antibody that binds to PD-1, e.g., human PD-1.

In an embodiment of the invention, the APCs are cultured in the presence of an activating substance such as Staphylococcal enterotoxin. In an embodiment of the invention the T-cell activating agent is a Staphylococcal enterotoxin such as Staphylococcal enterotoxin B (but not Staphylococcal enterotoxin B (SEB) if the T-cell is a Jurkat cell), Staphylococcal enterotoxin A (SEA), Staphylococcal enterotoxin D (SED) or Staphylococcal enterotoxin E (SEE).

In an embodiment of the invention, the ratio of T-cells (e.g., Jurkat cells) to antigen-presenting cells (e.g., Raji cells) is about 1/1, 2/1, 4/1, 8/1, 16/1 or 32/1 (e.g., 1-32). The present invention provides a method for determining if or to what extent a test substance stimulates T-cells:

(1)
(a) contacting a co-culture that comprises:
human or cynomolgous monkey T-cells (e.g., Jurkat cells) which express LAG3 and CD3 at a ratio of about 2/1 (LAG3/CD3) or lower, and
human or cynomolgous monkey antigen-presenting cells (e.g., Raji cells), with the test substance, in the presence of a T-cell activating agent, e.g., wherein the number of T-cells to APCs is at a ratio of about 4/1; and
(b) contacting a co-culture that comprises:
human or cynomolgous monkey T-cells (e.g., Jurkat cells) which express LAG3 and CD3 at a ratio of about 2/1 or lower, and
human or cynomolgous monkey antigen-presenting cells (e.g., Raji cells), with a substance that is known not to inhibit or agonize LAG3 or in the absence of any such substance or test substance, in the presence of a T-cell activating agent, e.g., wherein the number of T-cells to APCs is at a ratio of about 4/1; and
(2) determining the activation level of the T-cells of 1(a) and 1(b);
wherein the test substance is determined to immunostimulate T-cells if the T-cells of 1(a) are determined to be activated at a greater level than the T-cells of 1(b); and
wherein the test substance is determined to not immunostimulate or to immunosuppress T-cells if the T-cells of 1(a) are determined to be activated at no greater a level or a lower level than the T-cells of 1(b). The level of cytokine production by the T-cells of 1(a) relative to those of 1(b) indicates the extent to which the T-cells are activated.

The activation level of the T-cells can be determined by determining the secretion of a cytokine, such as IL-2, from the T-cells. In this embodiment, T-cells are determined to be activated to the extent that they secrete cytokine. Greater levels of cytokine production indicate a greater T-cell activation level.

In an embodiment of the invention, the method comprises the steps:

(1)
(i) incubating Raji cells with a T-cell activating agent;
(ii) incubating Jurkat cells expressing LAG3 and CD3 at a ratio of about 2/1 (LAG3/CD3) or lower with test substance, for example, incubating several samples of Jurkat cells each with one of a range of several concentrations of the test substance;
(iii) combining the Raji and Jukat cell preparations;
(iv) collecting the supernatant of the combined cells; and
(v) determining IL2 in the supernatants;
(2)
(i) preparing a suspension of Raji cells and incubating the Raji cells with a T-cell activating agent;
(ii) incubating Jurkat cells expressing LAG3 and CD3 at a ratio of about 2/1 (LAG3/CD3) or lower with a substance that is known not to inhibit or agonize LAG3 or in the absence of any such substance or test substance;
(iii) combining the Raji and Jukat cell preparations;
(iv) collecting the supernatant of the combined cells; and
(v) determining IL2 in the supernatants;
wherein the test substance is determined to immunostimulate T-cells if the T-cells of (1) are determined to be activated at a greater level than the T-cells of (2); and
wherein the test substance is determined to not immunostimulate or to immunosuppress T-cells if the T-cells of (1) are determined to be activated at no greater a level or a lower level than the T-cells of (2). The level of cytokine production by the T-cells of 1 relative to those of 2 indicates the extent to which the T-cells are activated.

In an embodiment of the invention, the method is characterized by one or more of any of the following:
Raji suspension having 2×10⁵ cells/ml;
Raji suspension in RPMI with 10% dialyzed fetal bovine serum (FBS);
T-cell activating agent is SED;
T-cell activating agent is SED at 120 ng/ml;
Raji cells and T-cell activating agent incubated for 30 minutes;
Raji cells incubated at 37° C.;
Jurkat cells at 8×10⁶ cells/ml,
Test substance is an anti-LAG3 antibody or antigen-binding fragment or LAG3 polypeptide or fragment thereof;
Multiple concentrations of at least 10 micrograms/ml of anti-LAG3 tested (e.g., 10-fold increases in concentration);
Raji and Jurkat cell preparations incubated together for 4 or 24 hours.

In an embodiment of the invention, methods discussed herein include the step of determining the ratio of LAG3 and CD3 expressed by the T-cells, for example, before employing the T-cells in the methods, e.g., before contacting T-cells with a test agent or with APCs. In an embodiment of the invention, such a step comprises determining that the ratio of LAG3/CD3 is 2 or lower and, if the ratio is two or lower, then proceeding with performing the remaining steps of the method. For example, in an embodiment of the invention, the expression levels of LAG3 and CD3 are determined by staining T-cells with anti-LAG3 and anti-CD3 antibodies and quantitating expression of each as a function of antibody staining intensity on the T-cells relative to that of a control antibody other than anti-LAG3 or anti-CD3 (e.g., an isotype antibody control). Staining intensity may be quantitated by fluorescence activated cell sorting (FACS) wherein the antibodies are stained (directly or indirectly) with a fluorescent agent.

T-cell activation can also be determined based upon the level of activation of an NFAT (Nuclear Factor of Activated T cells) responsive element or IL-2 promoter or a fragment thereof, e.g., which is operably linked to a reporter gene; e.g., a promoter that comprises one or more NFAT responsive elements, which is in the T-cell. This determination may be made, for example, instead of determining IL2 or cytokine production in the methods described herein. A reporter gene is any gene whose expression can be detected in a T-cell. In an embodiment of the invention, the reporter gene is not an NFAT or IL2. For example, in an embodiment of the invention, the T-cell activation is determined by determining luciferase reporter gene expression (e.g., Renilla or firefly luciferase). Reporter gene activation may be determined, for example, by lysing T-cells and analyzing the cell lysate for expression of the reporter gene (e.g., its encoded RNA, polypeptide or enzyme or any of the enzyme's products). In an embodiment of the invention, the reporter is operably linked to a minimal (m)CMV promoter and tandem repeats of the NFAT promoter consensus sequence. In an embodiment of the invention, the NFAT-luciferase reporter is a multimer of a core transcriptional response element (TRE). Typically, the TRE copy number is 4, 5 or 6 and drives expression of the reporter gene. Normally, the TRE responds to NFAT1, NFAT2, NFAT3 or NFAT4. In an embodiment of the invention, the NFAT transcriptional response element sequence is GGAGGAAAAACTGTTTCATACAGAAGGCGT (SEQ ID NO: 80). The level of reporter production in the T-cells indicates the extent to which such cells are activated.

A coding sequence is “under the control of”, “functionally associated with” or “operably associated with” or “operably linked to” transcriptional and translational control sequences in a cell when the sequences direct transcription of the coding sequence.

In an embodiment of the invention, the activation signal is expression of a reporter gene such as luciferase (e.g., Renilla, Vibrio or Photinus pyralis luciferase) or green fluorescent protein (GFP) from a reporter construct. In an embodiment of the invention, the reporter gene is operably linked to a promoter whose expression is induced when in a T-cell when the T-cell is activated. For example, in an embodiment of the invention, the promoter includes the NFAT (Nuclear Factor of Activated T cells) responsive element (NFAT-RE) or the IL-2 promoter or a fragment thereof. In an embodiment of the invention, the reporter construct comprises the reporter gene (e.g., luciferase or green fluorescent protein) operably linked to a minimal (m)CMV promoter and tandem repeats of the NFAT consensus sequence.

### EXAMPLES

These examples are intended to exemplify the present invention are not a limitation thereof. Compositions and methods set forth in the Examples form part of the present invention.

### Example 1: LAG3 Assay

The components of an immune synapse between a T-cell and an antigen presenting cell (APC) is required for LAG3 signaling. This T-cell and APC immune synapse can be recreated using the Jurkat cell line as a surrogate for a TCR expressing T-cell, the Raji cell line as a surrogate for an MHC class II expressing APC, and staphylococcal enterotoxin (SE) as an antigen. Jurkat cells have been shown to only respond to a subset of SEs with SEA, SEE, and SED showing the strongest responses as measured by secreted IL-2 (Table 1).

TABLE 1
IL-2 Production in response to toxin.

| | | None | SEA | SEB | SEC1 | SED | SEE | TSST | ExF |
|---|---|---|---|---|---|---|---|---|---|
| T cell | APC | U/ml | | | | | | | |
| Jurkat* | CH12.1ⁱ | <10 | <10 | <10 | <10 | <10 | 320 | <10 | <10 |
| (h Vβ8) | Raji | <10 | 160 | 40 | <10 | >640 | >640 | <10 | <10 |
| | DAP-DR1 | <10 | <10 | <10 | <10 | >640 | >640 | <10 | <10 |

Herman et at J Exp Med-v172, p709-17 (1990).

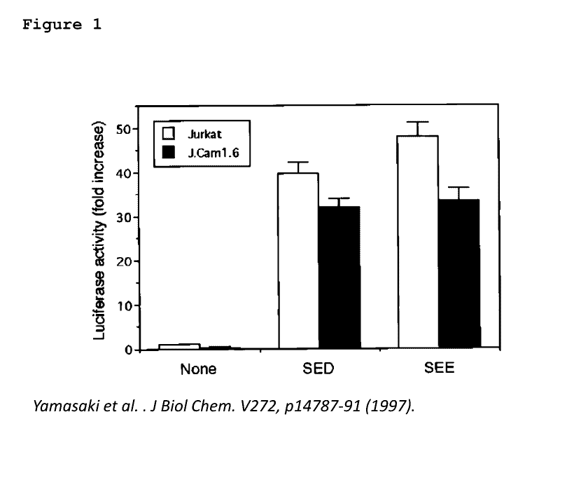

SED and SEE mediated stimulation of Jurkat cells co-cultured with Raji cells was also evaluated using an NFAT transcriptional element linked to a luciferase reporter gene (FIG. 1).

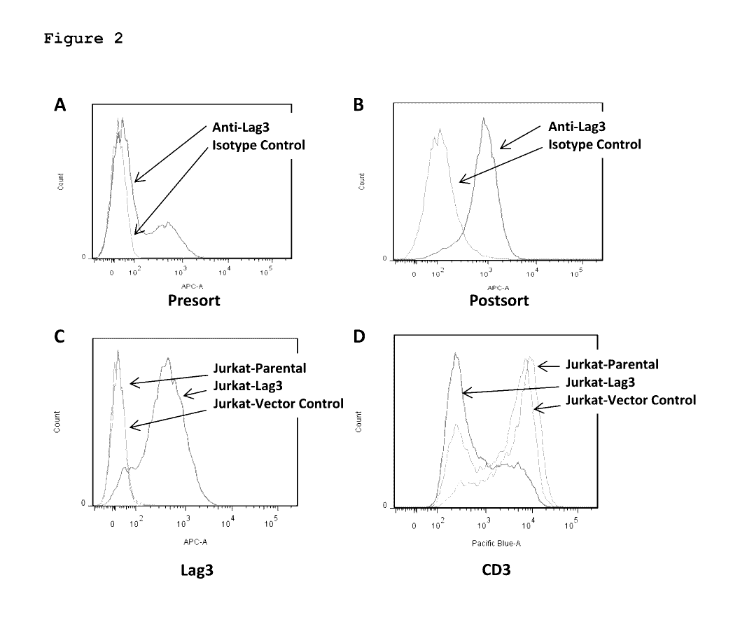

A human LAG3 transgene was introduced into Jurkat cells using a retroviral deliver system. LAG3 expression on the transduced Jurkat cells was low (FIG. 2A), therefore the cells were sorted for higher LAG3 expression (FIG. 2B). This enriched population of LAG3 Jurkat cells was subsequently stained for LAG3 and CD3 expression (FIGS. 2C and 2D). Compared to non-transduced parental cells and vector control transduced cells, LAG3 expressing Jurkat cells had a pronounced reduction in CD3 expression (FIG. 2D).

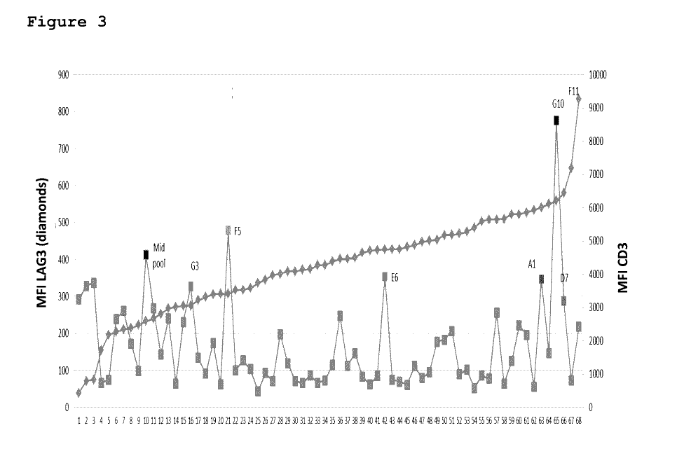

Because CD3 is a key part of the T-cell receptor (TCR) and immune synapse, clonal selection of LAG3 Jurkat cells was initiated to identify individual cell clones which maintained appropriate levels of both LAG3 and CD3 expression. Limiting dilution was used to generate 67 individual cell clones which were then stained for LAG3 (diamonds) and CD3 (squares) (FIG. 3). Clones G3, F5, E6, A1, D7, H3, G10 and F11 were chosen for subsequent analysis.

The eight selected Jurkat clones were re-stained for LAG3 and CD3 expression and TCR signaling was assessed using a stimulation cocktail of anti-CD28 and anti-CD3 antibodies (Table 2). TCR signaling was measured by secreted IL-2 and the range of response varied over 300-fold among the selected Jurkat clones. These results showed that a peak TCR response required the optimal balance of a robust level of CD3 expression, an intermediate level of LAG3 expression, and a ratio of LAG3 to CD3 that is 2-fold or lower. Based on these criteria, clone G10 was selected for further analysis and assay development.

TABLE 2
Profile of Jurkat clones.

| | Lag3 | CD3 | | |
|---|---|---|---|---|
| | Expression | expression | Secreted IL2 | Ratio of |
| Clone ID | (Fold Change) | (Fold Change) | (Fold Change)* | Lag3/CD3 |
| Clone G10 | 16.7 | 8.4 | 1143.5 | 2.0 |
| Clone G3 | 17.8 | 5.3 | 101.6 | 3.3 |
| Clone F5 | 20.1 | 7.5 | 48.3 | 2.7 |
| Clone E6 | 28.3 | 8.5 | 30.1 | 3.3 |
| Clone F11 | 38.0 | 3.9 | 10.1 | 9.9 |
| Clone H3 | 14.5 | 5.0 | 4.5 | 2.9 |
| Clone A1 | 12.3 | 3.5 | 4.1 | 3.6 |
| Clone D7 | 26.7 | 3.4 | 3.8 | 7.8 |

*500 ng/ml anti-CD3 + 250 ng/ml anti-CD28 for 24 hrs

Cell suspensions from the clones (scaled up to 24 wells) were incubated with either stain mix (maHuCD3 pacblue (MOPC-21; BP Pharmigen))+Humanized aLag3-DyLight650 (SEQ ID NOs: 84 and 85)) or control mix (mIgG1 pacblue (MOPC-21; IgG1/κ)+Herceptin-DyLight 650). Cells were incubated for 30 minutes with shaking at 4 degrees Celcius. Cells were washed with PBS-BSA buffer and fixed with 2% paraformaldehyde. Samples acquired using the FACS cell sorter (BD FACSCanto).

In Table 2 and Table 3, LAG3 and CD3 expression fold change refers to FACS staining of cells with anti-LAG3 antibody or anti-CD3 antibody relative to staining with a control antibody.

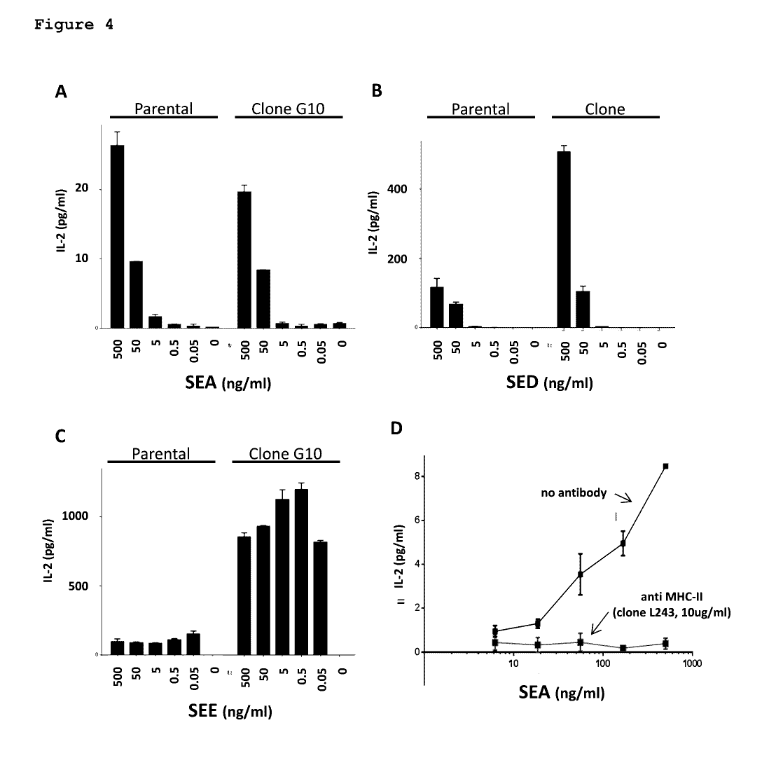

Jurkat LAG3 clone G10 was co-cultured with Raji cells in the presence of three forms of staphylococcal enterotoxin starting at 500 ng/ml and titrating down to 0.05 ng/ml. As previously shown by Herman et al., SEA elicited a moderate stimulatory effect (FIG. 4A) whereas SED (FIG. 4B) and SEE (FIG. 4C) were much stronger antigens. Interestingly, clone G10 showed a much stronger stimulatory response to SED and SEE than the parental Jurkat cells which may have been reflective of clone G10 having an optimal CD3 and being selected for robust IL-2 response. To demonstrate that MHC class II was mediating the stimulatory effects of the staphylococcal enterotoxins, the SE stimulation was performed in the presence of a MHC class II antibody which effectively blocked the SEA simulation of IL-2 secretion in the assay (FIG. 4D).

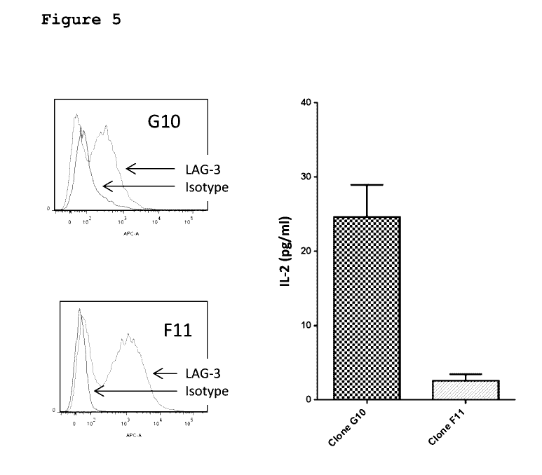

Clone G10 was compared to Clone F11 which had higher levels of LAG3 and lower levels of CD-3 (Table 2). These Jurkat clones were re-stained for LAG3 expression and then co-cultured with Raji cells in the presence of 100 ng/ml of SED. FIG. 5 showed clone G10 responded much more robustly to SED stimulation suggesting that the high levels of LAG3 and lower levels of CD3 in clone F11 were deleterious for an optimal response to SED stimulation.

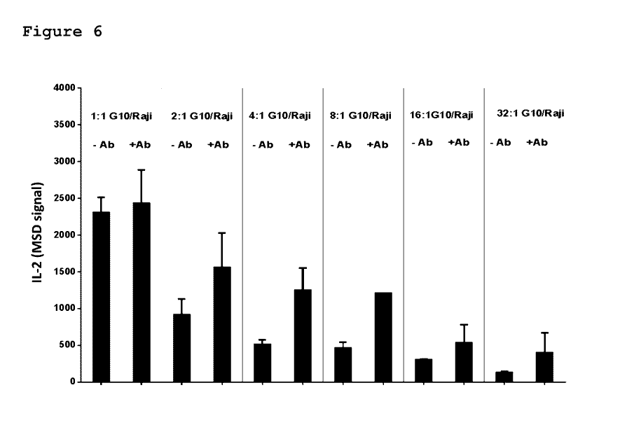

To identify the optimal amount of MHC class II present in the assay, a fixed number of Jurkat clone G10 cells (100,000) were co-cultured with a variety of Raji cell ratios ranging from equal numbers of Jurkat and Raji to 32:1 Jurkat to Raji ratio (FIG. 6). As the relative amounts of MHC class II are reduced by the presence of fewer Raji cells, the IL-2 secretion stimulated by 500 ng/ml of SEA is greatly reduced. An antibody targeting LAG3 was added to these experiments at 10 ug/ml (+Ab) to identify the optimal ratio of Jurkat clone G10 and Raji cells that will form an immune synapse that responds to SEA but is still capable of showing an additional IL-2 response when the anti-LAG3 antibody binds and blocks the immunosuppressive function of LAG3. Based on these experiments, a ratio of 4:1 Jurkat to Raji was selected.

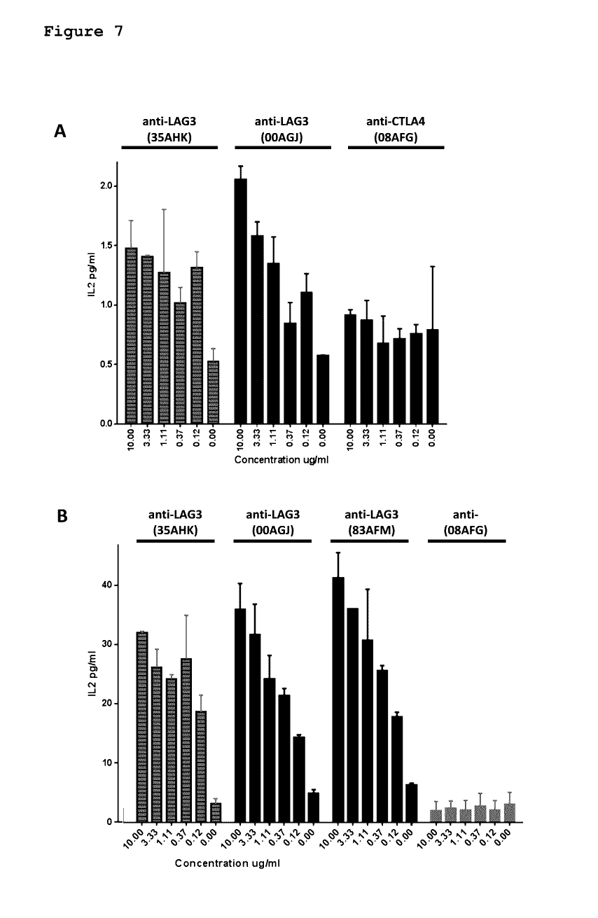

As discussed in FIG. 6, SEA was an effective superantigen and, under optimized assay conditions, anti-LAG3 has been shown to relieve the repressive function of LAG3. However, the amount of IL-2 secreted in these assays was in the 0.5-2 pg/ml range which presented challenges for signal to noise ratios and dynamic range for the fold change of anti-LAG3 treatment. Therefore, the Jurkat bioassay was setup with 500 ng/ml of SEA (FIG. 7A) and compared to treatment with 50 ng/ml of SED (FIG. 7B). Stimulation with SED resulted in significantly higher levels of IL-2 production which then allowed for an improved signal to noise ratio and dose-response for anti-LAG3 antibody treatments.

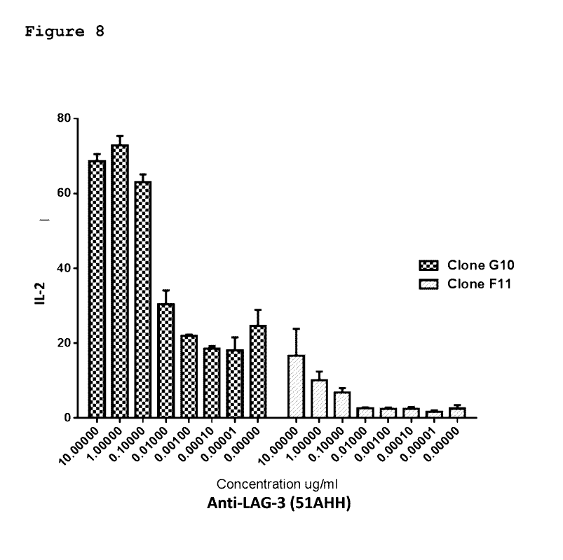

Anti-LAG3 dose responses were evaluated in Jurkat clone G10 and compared to clone F11. As discussed in Table 2 and FIG. 5, clone F11 had a non-ideal level of both CD3 and LAG3 expression and this was reflected in the observed dose response to anti-LAG3 treatment (FIG. 8). While clone F11 did exhibit a limited dose response to anti-LAG3, the response observed for clone G10 was more pronounced with a superior signal to noise ratio. This level of response again highlighted the value of selecting clone G10 on the basis of an optimal ratio of LAG3 and CD3 expression where LAG3 expression was not too high, CD3 expression was not too low, and the ratio between them was approximately 2-fold or lower.

Six separate experiments with Jurkat clone G10 cells were conducted and anti-LAG3 dose responses were evaluated and compared with CD3 and LAG3 expression levels (Table 3). Changes in LAG3 and CD3 expression were observed in the different experiments and are likely attributable to cell passage number and culturing conditions. When the ratio of LAG3 to CD3 was between 0.6 and 1.6, the fold change in secreted IL-2 in response to anti-LAG3 treatment varied from 5.2 to 11.2 fold. Importantly, when the LAG3 to CD3 ratio was 0.2, the fold change for anti-LAG3 treatment dropped to 3.2 fold. Taken together with the other Jurkat clones profiled in Table 2, this data suggests that the optimal ratio for LAG3 to CD3 expression is less than 2 fold and greater than 0.2 fold.

TABLE 3
Characterization of Jurkat clone G10 after various numbers of passages.

| | | | | | Secreted IL-2 | |
|---|---|---|---|---|---|---|
| | | Lag3 | CD3 | | (Fold-Change | |
| | | Expression | Expression | | after MK- | |
| | Experiment | (Fold | (Fold | Ratio of | 4280 | |
| Clone ID | Number | Change) | Change) | Lag3/CD3 | treatment) | Passage |
| Clone G10 | 1 | 2.0 | 8.0 | 0.2 | 3.2 | P6 |
| Clone G10 | 2 | 6.1 | 10.9 | 0.6 | 5.7 | ~P3 |
| Clone G10 | 3 | 5.6 | 7.2 | 0.8 | 6.9 | P4 |
| Clone G10 | 4 | 7.2 | 6.9 | 1.0 | 5.2 | P14 |
| Clone G10 | 5 | 7.3 | 6.4 | 1.1 | 6.3 | P12 |
| Clone G10 | 6 | 7.3 | 4.7 | 1.6 | 11.2 | P17 |

Cell suspensions (2×10⁶ cells/ml) from the Clone G10 that was maintained in culture were incubated with either maHuCD3 pacblue or mIgG1-pacblue (MOPC-21; BD Pharmigen) to determine CD3 expression. The cells were stained with DyLight 650 labelled Ab6 (51AHH) or control antibody Herceptin-DyLight 650 to determine LAG3 expression. Cells were incubated for 30 minutes with shaking at 4 degrees Celcius. Cells were washed with PBS-BSA buffer and fixed with 2% paraformaldehyde. Samples acquired using the FACS cell sorter (BD FACSCanto).

There is a loose correlation between passage number and LAG3/CD3 ratio. The differences in LAG3 expression observed between Table 2 and Table 3 may be due to the fact that the Table 2 data was from initial characterization of clone G10 immediately after it had been isolated. Expression levels can change and stabilize once a cell clone is established and a frozen stock has been banked. Alternatively, the staining of LAG3 in Table 2 used a different antibody than the staining used to generate the LAG3 expression data in Table 3. Table 3 and all subsequent work was conducted with a directly labeled anti-LAG3 antibody. Differences in detection mAb for staining could also explain differences in expression.

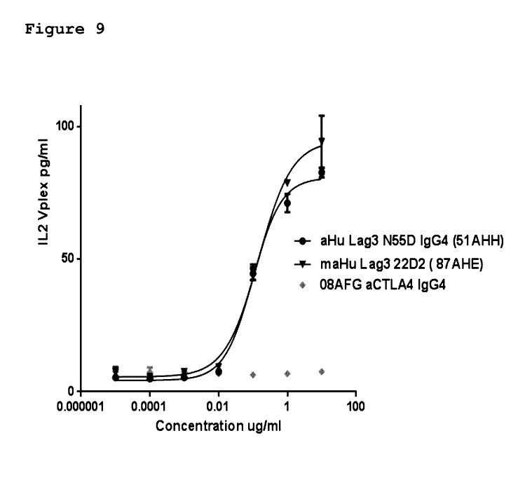

The LAG3 bioassay represented the first in vitro human cell line assay that recapitulated the optimal balance of components of an immune synapse required to observe LAG3-mediated repression of the MHC class II and TCR interaction and anti-LAG3 mediated relief of that repression (FIG. 9). The key components included a Jurkat clone, G10, engineered to overexpressing LAG3 and then selected for optimal LAG3 and CD3 expression levels, MCH class II expressing Raji cells present at a specific ratio and antigen stimulation via SED at a sub-maximal concentration. Using these conditions, the LAG3 bioassay delivered high signal to noise ratios, a dynamic range of approximately 10-fold, and reproducible EC50 values to support potency comparisons for non-clinical pharmacology studies and CMC analytical potency release testing.

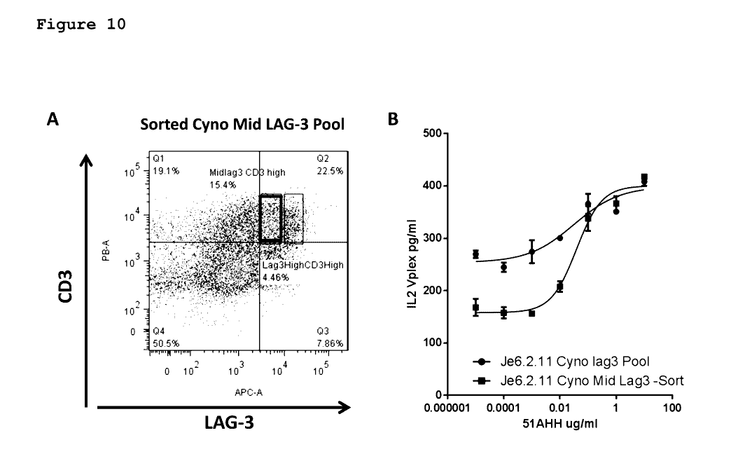

LAG3 expression on primary cells isolated from PBMCs from human and non-human primates was very low which presented challenges in demonstrating bioactivity and potency for anti-LAG3 antibodies. To date, these challenges have prevented the development of a suitable bioassay for evaluating bioactivity of anti-LAG3 antibodies on cynomolgus LAG3 expressing cells. Using lessons learned from the development of an optimized Jurkat human LAG3 bioassay, a cynomolgus LAG3 transgene was introduced into the Jurkat cell line. The pool of cynomolgus LAG3 transduced Jurkat cells had a wide range of LAG3 and CD3 expression (FIG. 10A). Based on experience with the human bioassay, cynomolgus LAG3 Jurkat cells were sorted (box) for a population where LAG3 and CD3 expression levels were approximately equivalent—targeting the ratio of 1:1 to 2:1 that was found ideal for the human LAG3 assay. A comparison of anti-LAG3 treatment of these two pools of cynomolgus LAG3 Jurkats in the bioassay format demonstrated that the sorted population of cynomolgus LAG3 Jurkat cells with an optimal ratio of LAG3:CD3 performed superior to the pool of cynomolgus LAG3 Jurkat cells with a broader expression range of LAG3:CD3 (FIG. 10B). This finding reinforced the observation that there was an ideal ratio of LAG3:CD3 for optimal assay performance. The Raji cells, SED, and other conditions were the same as the human bioassay.

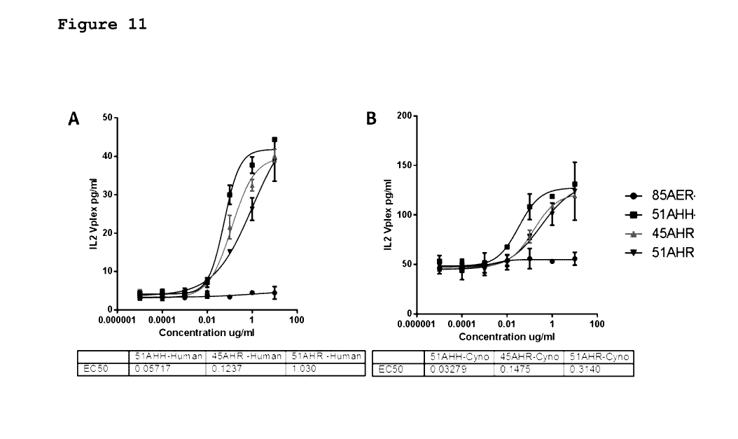

Three different anti-LAG3 antibodies (51AHH: humanized anti-human LAG3 VH6 N55D/VL3, IgG4 S228P/Kappa, 45AHR: human anti-human LAG3, IgG4 S228P/Kappa, 51AHR: human anti-human LAG3, IgG4 S228P/kappa) were compared in the human bioassay (FIG. 11A) and the cynomolgus bioassay (FIG. 11B). The potency values and rank order of these mAbs were comparable across species. 85AER (humanized anti-RSV, hinge mutation S228P IgG4) was a control and had no activity in these assays.

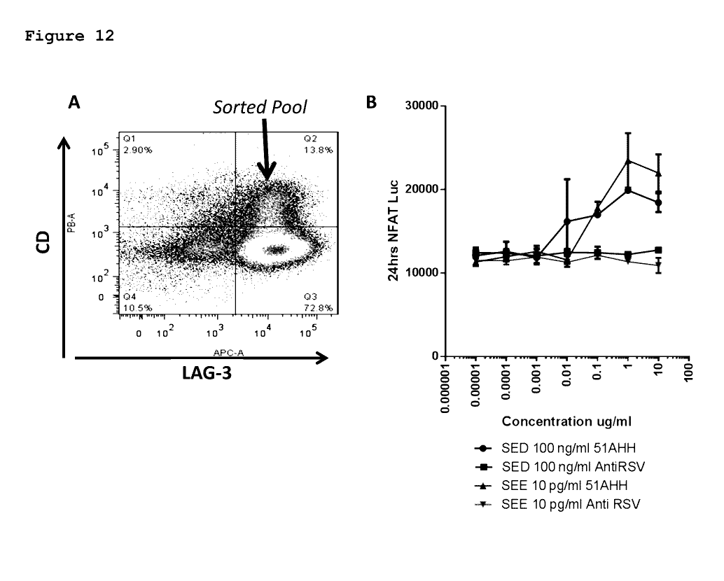

The LAG3 bioassay relied on secreted IL-2 as a readout. An NFAT reporter system was also evaluated. Jurkat cells were transduced with lentivirus constructs containing human LAG3 and NFAT-luciferase. As before, LAG3 and CD3 expression was evaluated and a sub-population of cells was sorted and selected based on an approximate 1:1 to 2:1 ratio of expression (FIG. 12A). The sorted pool of Jurkat LAG3 NFAT-luciferase cells were co-cultured with Raji cells and stimulated with 100 ng/ml SED or 10 pg/ml SEE (FIG. 12B). In the presence of humanized anti-human LAG3 VH6 N55D/VL3, IgG4 S228P/Kappa treatment (51AHH), there was a dose-dependent increase in NFAT signaling which was indicative of the antibody blocking LAG3's inhibitory function and relieving LAG3 repression.

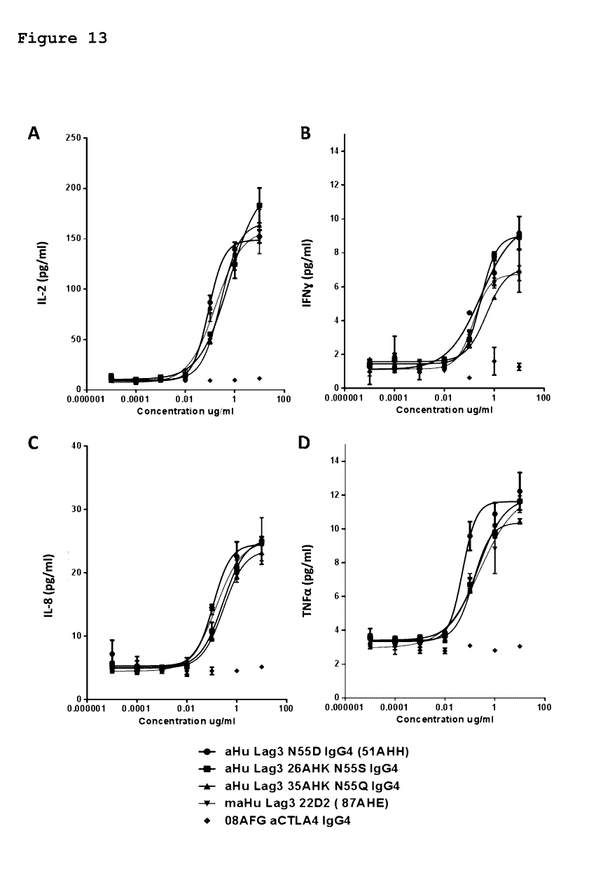

The Jurkat LAG3 bioassay was assessed for whether secreted cytokines, other than IL-2, were being modulated. A total of 10 cytokines were evaluated: IFN-γ, IL-1β, IL-2, IL-4, IL-6, IL-8, IL-10, IL-12p70, IL-13, and TNF-α. IL-2 responses to anti-LAG3 treatment showed the antibody dose dependent relief of LAG3 repression that was typical for this assay (FIG. 13A). Interestingly, interferon-γ (FIG. 13B), interleukin-8 (FIG. 13C), and tumor necrosis factor alpha (FIG. 13D) all showed dose-responsive increases upon anti-LAG3 antibody treatment.

### Materials and Methods.

### Assay Protocol: Cyno and Human LAG3 Potency Assay

Prepared a Raji cell suspension (2×10⁵ cells/ml) in RPMI media containing 10% dialyzed FBS. Incubated the Raji cells with 120 ng/ml of SED toxin for 30 minutes in a 37° C. incubator. Simultaneously incubated a cell suspension of Jurkat cells, 8×10⁶ cells/ml (In-house Clone G10-expressing Human LAG3 OR Mid pool-expressing cyno LAG3) with a log fold titration of (starting at 10 ug/ml) anti-LAG3 or control antibodies. Added the SED loaded Raji cells to the cells incubated with antibody for 24 hours in a 37° C. incubator (i.e., Raji and Jurkat cells were incubated together for 24 hours at 37° C.). Collected supernatants and analyzed using the 1L2 V plex kit or a 10 plex from MSD (sandwich immunoassays which use electrochemiluminescent labels conjugated to detection antibodies (MSD=mesoscale device)).

### Assay Protocol: Human Lac:13 NFAT Reporter Assay:

Serum starved the Jurkat cells expressing LAG3 overnight in optimum+1% dialyzed FBS. Prepared a Raji cell suspension (1.66×10⁵ cells/ml) in RPMI media containing 10% dialyzed FBS. Incubated the Raji cells with 133 ng/ml of SED toxin for 30 minutes in a 37° C. incubator. Simultaneously incubated a cell suspension of Jurkat cells, 8×10⁶ cells/ml (Je6.2.11-Lag3NFAT-Luc) with a log fold titration of (starting at 10 ug/ml) anti-LAG3 or control antibodies. Added the SED loaded Raji cells to the cells incubated with antibody for 24 hours in a 37° C. incubator. At the end of 24 hours, luciferase activity was measured using 100 ul of One Glo Promega reagent and an Envision plate reader.

### REFERENCES

Freeman G J, Sharpe A H. A new therapeutic strategy for malaria: targeting T cell exhaustion. Nat Immunol. 2012 Jan. 19; 13(2):113-5.
Goldberg M V, Drake C G. LAG3 in Cancer Immunotherapy. Curr Top Microbiol Immunol. 2011; 344:269-78.
Herman A, Croteau G, Sekaly R P, Kappler J, Marrack P. HLA-DR alleles differ in their ability to present staphylococcal enterotoxins to T cells. J Exp Med. 1990 Sep. 1; 172(3):709-17.
Sierro S, Romero P, Speiser D E. The CD4-like molecule LAG3, biology and therapeutic applications. Expert Opin Ther Targets. 2011 January; 15(1):91-101.
Yamasaki S, Tachibana M, Shinohara N, Iwashima M. Lck-independent triggering of T-cell antigen receptor signal transduction by staphylococcal enterotoxins. J Biol Chem. 1997 Jun. 6; 272(23): 14787-91.

### Example 2: Bifunctional PD1/LAG3 Bioassay

This system accommodates other immunomodulatory receptors to assess the combination benefit of simultaneously modulating two or more receptors. Using lentiviral transduction, a human PD1 (Programmed Death-1) transgene was introduced into the Lag3 overexpressing DT1088-G10 clone and a human PD-L1 (Programmed Death Ligand-1) transgene was introduced into the Raji cells. The DT1088G10-PD1 cells were sorted for maximal expression of PD-1. See Table 3 regarding the balance of LAG3 and CD3 expression for G10. Raji-PDL1 cells were sorted for maximal expression of PD-L1 and MHCII. For the assay, 100,000 DT1088G10-PD1 Jurkat cells were co-cultured with 100,000 Raji-PDL1 cells that are preloaded with 100 ng/ml SED toxin in the presence of different antibody titrations. Supernatants were collected and secreted IL2 levels measured using a MSD V-plex kit after 24 hour of antibody treatment.

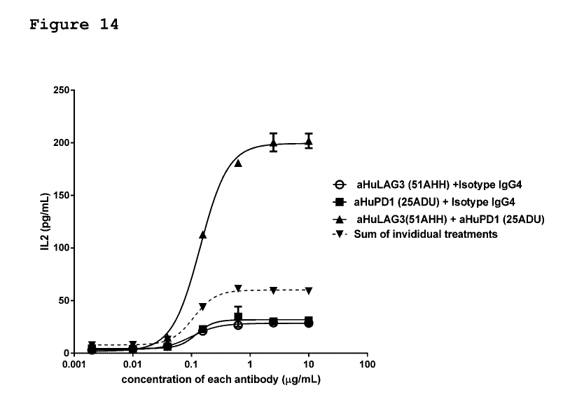

The impact of combining anti-human LAG3 antibody and anti-human PD1 was assessed (FIG. 14). A matched IgG4 isotype antibody (anti-human PCSK9) was used as a negative control and combined with either anti-human LAG3 or anti-human PD1 so that each treatment group had the same overall amount of antibodies. Antibody treatment was a combination of two antibodies—starting at 10 ug/ml for each. Treatment with anti-human LAG3 resulted in a 10-fold dose response and treatment with anti-human PD1 had a 9-fold change which demonstrated the individual components of both LAG3 and PD1 signaling have been reconstituted in this bioassay platform. The dotted line demonstrated the expected IL2 levels if the individual anti-human LAG3 and anti-human PD1 responses were summed, resulting in a 19-fold change. However, when anti-human LAG3 and anti-human PD1 were actually combined together in this bioassay, the combined effect of blocking both PD1 and LAG3 pathways revealed a 54-fold enhancement in IL2 production.

By developing a bi-functional assay for LAG3 and PD1, this platform is capable of reading out single agent responses as well as demonstrating the value of simultaneous blockade of both PD1 and LAG3. This assay is value for, inter alia, screening and functional assessment of combinations of antibodies, nanobodies, and bispecific molecules.

The present invention is not to be limited in scope by the specific embodiments described herein. Indeed, the scope of the present invention includes embodiments specifically set forth herein and other embodiments not specifically set forth herein; the embodiments specifically set forth herein are not necessarily intended to be exhaustive. Various modifications of the invention in addition to those described herein will become apparent to those skilled in the art from the foregoing description. Such modifications are intended to fall within the scope of the claims.

Patents, patent applications, publications, product descriptions, and protocols are cited throughout this application, the disclosures of which are incorporated herein by reference in their entireties for all purposes.

We claim:

1. An isolated human or cynomolgous monkey T-cell which expresses human or cynomolgous monkey LAG3 and human or cynomolgous monkey CD3 at a ratio of about 2/1 (LAG3/CD3) or lower.

2. The cell of claim 1 the ratio is:
$$\frac{M F I T \text{-} cell ( anti \text{-} L A G 3 ) / M F I T \text{-} cell ( L A G 3 control )}{M F I T \text{-} cell ( anti \text{-} CD 3 ) / M F I T \text{-} cell ( CD 3 control )} ;$$
wherein,
MFI T-cell (anti-LAG3) is the mean fluorescent intensity observed in fluorescence activated cell sorting (FACS) analysis of the T-cell stained with the Ab6 (IgG4/κ) anti-LAG3 antibody that is labeled with DyLight650,
MFI T-cell (LAG3 control) is the mean fluorescent intensity observed in fluorescence activated cell sorting (FACS) analysis of the T-cell stained with trastuzumab that is labeled with DyLight 650;
MFI T-cell (anti-CD3) is the mean fluorescent intensity observed in fluorescence activated cell sorting (FACS) analysis of the T-cell stained with the maHuCD3 pacblue anti-CD3 antibody; and
MFI T-cell (CD3 control) is the mean fluorescent intensity observed in fluorescence activated cell sorting (FACS) analysis of the T-cell stained with mIgG1-pacblue.

3. The cell of claim 1 which is Jurkat, CCRF-CEM; HPB-ALL; HPB-MLT; HD-Mar-2; TALL-I; MOLT-16, MAT; H9; ED-S; or ATL-35T.

4. The cell of claim 1 which further expresses PD-1.

5. A composition comprising the cell of claim 1 and a human or cynomolgous monkey antigen-presenting cell wherein the composition comprising the T-cell and the antigen-presenting cell is a single vessel or are in separate vessels.

6. The composition of claim 5 wherein the antigen-presenting cell expresses PD-L1.

7. The composition of claim 5 wherein the antigen-presenting cell is a cancerous cell.

7. The composition of claim 5 wherein the antigen-presenting cell is a Raji cell, Daudi cell, JY cell, melanoma cell, L-cell that overexpresses HLA-DR, or HLA-DR B7.

8. The composition of claim 5 comprising a T-cell activating agent.

9. The composition of claim 5 wherein the T-cell activating agent is a Staphylococcal enterotoxin that is not Staphylococcal enterotoxin B (SEB) if the T-cell is a Jurkat cell.

10. The composition of claim 5 comprising an antibody or antigen-binding fragment that specifically binds to human or cynomolgous monkey LAG3 or PD-1.

11. The composition of claim 5 wherein the antibody or antigen-binding fragment thereof comprises:
CDRs comprising the amino acid sequences set forth in: SEQ ID NOs: 3-8;
CDRs comprising the amino acid sequences set forth in: SEQ ID NOs: 3, 4, 5, 6, 11 and 8;
CDRs comprising the amino acid sequences set forth in: SEQ ID NOs: 3, 4, 5, 6, 14 and 8;
CDRs comprising the amino acid sequences set forth in: SEQ ID NOs: 3, 4, 5, 6, 17 and 8;
CDRs comprising the amino acid sequences set forth in: SEQ ID NOs: 3, 4, 5, 6, 28 and 8;
CDRs comprising the amino acid sequences set forth in: SEQ ID NOs: 3, 4, 5, 6, 29 and 8;
CDRs comprising the amino acid sequences set forth in: SEQ ID NOs: 30-35;
CDRs comprising the amino acid sequences set forth in: SEQ ID NOs: 36-41;
CDRs comprising the amino acid sequences set forth in: SEQ ID NOs: 42-47;
CDRs comprising the amino acid sequences set forth in: SEQ ID NOs: 48-53;
CDRs comprising the amino acid sequences set forth in: SEQ ID NOs: 54-59;
CDRs comprising the amino acid sequences set forth in: SEQ ID NOs: 60-65;
CDRs comprising the amino acid sequences set forth in: SEQ ID NOs: 66-71;
CDRs comprising the amino acid sequences set forth in: SEQ ID NOs: 72-77;
a light chain immunoglobulin comprising the amino acid sequence set forth in SEQ ID NO: 1 or
a variable domain thereof and a heavy chain immunoglobulin comprising the amino acid sequence set forth in SEQ ID NO: 2 or a variable domain thereof;
a light chain immunoglobulin comprising the amino acid sequence set forth in SEQ ID NO: 9 or a variable domain thereof and a heavy chain immunoglobulin comprising the amino acid sequence set forth in SEQ ID NO: 10 or a variable domain thereof;
a light chain immunoglobulin comprising the amino acid sequence set forth in SEQ ID NO: 12 or a variable domain thereof and a heavy chain immunoglobulin comprising the amino acid sequence set forth in SEQ ID NO: 13 or a variable domain thereof;
a light chain immunoglobulin comprising the amino acid sequence set forth in SEQ ID NO: 15 or a variable domain thereof and a heavy chain immunoglobulin comprising the amino acid sequence set forth in SEQ ID NO: 16 or a variable domain thereof;
a light chain immunoglobulin comprising the amino acid sequence set forth in SEQ ID NO: 18 or a variable domain thereof and a heavy chain immunoglobulin comprising the amino acid sequence set forth in SEQ ID NO: 19 or a variable domain thereof;
a light chain immunoglobulin comprising the amino acid sequence set forth in SEQ ID NO: 20 or a variable domain thereof and a heavy chain immunoglobulin comprising the amino acid sequence set forth in SEQ ID NO: 21 or a variable domain thereof;
a light chain immunoglobulin comprising the amino acid sequence set forth in SEQ ID NO: 22 or a variable domain thereof and a heavy chain immunoglobulin comprising the amino acid sequence set forth in SEQ ID NO: 23 or a variable domain thereof;
a light chain immunoglobulin comprising the amino acid sequence set forth in SEQ ID NO: 24 or a variable domain thereof and a heavy chain immunoglobulin comprising the amino acid sequence set forth in SEQ ID NO: 25 or a variable domain thereof; or
a light chain immunoglobulin comprising the amino acid sequence set forth in SEQ ID NO: 26 or a variable domain thereof and a heavy chain immunoglobulin comprising the amino acid sequence set forth in SEQ ID NO: 27 or a variable domain thereof.

12. The composition of claim 5 comprising human or cynomolgous monkey T-cells and human or cynomolgous monkey antigen-presenting cells at a ratio of about 4/1.

13. A method for determining if or to what extent a test substance stimulates T-cells comprising:
contacting a co-culture that comprises human or cynomolgous monkey T-cells which express LAG3 and CD3 at a ratio of about 2/1 (LAG3/CD3) or lower, and, optionally, PD-1, and human or cynomolgous monkey antigen-presenting cells, which, optionally, express PD-L1, in the presence of a T-cell activating agent, with the test substance; and monitoring secretion of a cytokine from the T-cells or monitoring expression of a reporter in said T-cells that is operably linked to an IL2 promoter or to a promoter comprising the Nuclear Factor of Activated T-cells Response Element (NFAT-RE); wherein the test substance is determined to stimulate the T-cells if the T-cells secrete more cytokine or express more reporter in the presence of the test substance than in the absence of the test substance, and wherein the level of cytokine production or reporter expression by the T-cells indicates the extent to which the T-cells are activated.

14. The method of claim 13 wherein the test substance is an antibody or antigen-binding fragment thereof that binds specifically to LAG3 and/or PD-1.

15. The method of claim 13 wherein the antibody or antigen-binding fragment thereof binds to LAG3 and comprises:
CDRs comprising the amino acid sequences set forth in: SEQ ID NOs: 3-8;
CDRs comprising the amino acid sequences set forth in: SEQ ID NOs: 3, 4, 5, 6, 11 and 8;
CDRs comprising the amino acid sequences set forth in: SEQ ID NOs: 3, 4, 5, 6, 14 and 8;
CDRs comprising the amino acid sequences set forth in: SEQ ID NOs: 3, 4, 5, 6, 17 and 8;
CDRs comprising the amino acid sequences set forth in: SEQ ID NOs: 3, 4, 5, 6, 28 and 8;
CDRs comprising the amino acid sequences set forth in: SEQ ID NOs: 3, 4, 5, 6, 29 and 8;
CDRs comprising the amino acid sequences set forth in: SEQ ID NOs: 30-35;
CDRs comprising the amino acid sequences set forth in: SEQ ID NOs: 36-41;
CDRs comprising the amino acid sequences set forth in: SEQ ID NOs: 42-47;
CDRs comprising the amino acid sequences set forth in: SEQ ID NOs: 48-53;
CDRs comprising the amino acid sequences set forth in: SEQ ID NOs: 54-59;
CDRs comprising the amino acid sequences set forth in: SEQ ID NOs: 60-65;
CDRs comprising the amino acid sequences set forth in: SEQ ID NOs: 66-71;
CDRs comprising the amino acid sequences set forth in: SEQ ID NOs: 72-77;
a light chain immunoglobulin comprising the amino acid sequence set forth in SEQ ID NO: 1 or a variable domain thereof and a heavy chain immunoglobulin comprising the amino acid sequence set forth in SEQ ID NO: 2 or a variable domain thereof;
a light chain immunoglobulin comprising the amino acid sequence set forth in SEQ ID NO: 9 or a variable domain thereof and a heavy chain immunoglobulin comprising the amino acid sequence set forth in SEQ ID NO: 10 or a variable domain thereof;
a light chain immunoglobulin comprising the amino acid sequence set forth in SEQ ID NO: 12 or a variable domain thereof and a heavy chain immunoglobulin comprising the amino acid sequence set forth in SEQ ID NO: 13 or a variable domain thereof;
a light chain immunoglobulin comprising the amino acid sequence set forth in SEQ ID NO: 15 or a variable domain thereof and a heavy chain immunoglobulin comprising the amino acid sequence set forth in SEQ ID NO: 16 or a variable domain thereof;
a light chain immunoglobulin comprising the amino acid sequence set forth in SEQ ID NO: 18 or a variable domain thereof and a heavy chain immunoglobulin comprising the amino acid sequence set forth in SEQ ID NO: 19 or a variable domain thereof;
a light chain immunoglobulin comprising the amino acid sequence set forth in SEQ ID NO: 20 or a variable domain thereof and a heavy chain immunoglobulin comprising the amino acid sequence set forth in SEQ ID NO: 21 or a variable domain thereof;
a light chain immunoglobulin comprising the amino acid sequence set forth in SEQ ID NO: 22 or a variable domain thereof and a heavy chain immunoglobulin comprising the amino acid sequence set forth in SEQ ID NO: 23 or a variable domain thereof;
a light chain immunoglobulin comprising the amino acid sequence set forth in SEQ ID NO: 24 or a variable domain thereof and a heavy chain immunoglobulin comprising the amino acid sequence set forth in SEQ ID NO: 25 or a variable domain thereof; or
a light chain immunoglobulin comprising the amino acid sequence set forth in SEQ ID NO: 26 or a variable domain thereof and a heavy chain immunoglobulin comprising the amino acid sequence set forth in SEQ ID NO: 27 or a variable domain thereof.

16. The method of claim 13 wherein the cytokine is a member selected from the group consisting of IL-2, interferon gamma (IFNγ), IL-8 and tumor necrosis factor alpha (TNFα).

17. The method of claim 13 wherein expression of a reporter gene that is operably linked to a promoter comprising the Nuclear Factor of Activated T-cells Response Element (NFAT-RE) is measured to determine whether the T-cells are activated; wherein the T-cells are determined to be activated if expression of the reporter gene increases in the presence of the test substance relative to in the absence of the test substance.

18. The method of claim 13 wherein the T-cell is human.

19. The method of claim 13 wherein the T-cell is Jurkat, CCRF-CEM; HPB-ALL; HPB-MLT; HD-Mar-2; TALL-I; MOLT-16, MAT; H9; ED-S; or ATL-35T.

20. The method of claim 13 wherein the antigen presenting cell is a cancerous cell.

21. The method of claim 13 wherein the antigen-presenting cell is a Raji cell, Daudi cell, a JY cell, a melanoma cell, an L-cell that overexpresses HLA-DR, or HLA-DR B7.

22. The method of claim 13 wherein the T-cell activating agent is Staphylococcal enterotoxin that is not Staphylococcal enterotoxin B (SEB) if the T-cell is a Jurkat cell.

23. The method of claim 13 wherein the human or cynomolgous monkey T-cells and human or cynomolgous monkey antigen-presenting cells are at a ratio of about 4/1.

24. A method for making the composition of claim 5 comprising combining the T-cells and the antigen-presenting cells.

25. A composition that is the product of the method of claim 24.

26. A method for making the cell of claim 1 comprising introducing a polynucleotide encoding LAG3 into T-cells, determining which T-cells express LAG3 and CD3 at a ratio of about 2/1 (LAG3/CD3) or lower and selecting one or more T-cells exhibiting said ratio.

27. A cell that is the product of the method of claim 26.
